(19) United States
(12) Patent Application Publication
(10) Pub. No.: US 2020/0016315 A1
(43) Pub. Date: Jan. 16, 2020

(54) PURIFICATION OF BLOOD SAMPLES WITH HEMOLYSIS

(71) Applicant: Syddansk Universitet, Ddense M (DK)

(72) Inventors: Niels Jonas Heilskov Graversen, Odense C (DK); Soren Kragh Moestrup, Aarhus N (DK); Kirstine Lindhardt Saederup, Odense SØ (DK)

(21) Appl. No.: 16/498,256

(22) PCT Filed: Mar. 23, 2018
(86) PCT No.: PCT/EP2018/057534

§ 371 Date: Sep. 26, 2019

(30) Foreign Application Priority Data
Mar. 21, 2017 (EP) 17164080.8

(51) Int. Cl. A61M 1/34; G01N 1/34; G01N 33/72

(52) U.S. Cl. CPC A61M 1/3486; G01N 33/721; G01N 1/34

(57) ABSTRACT

The present invention relates to removal of hemoglobin from a sample. In particular the present invention relates to a method in which a protein with specificity towards hemoglobin and/or the complex of hemoglobin with haptoglobin is immobilized on a solid support and utilized to remove hemoglobin from a sample comprising hemoglobin, such as a hemolysed sample obtained from a subject.

### TECHNICAL FIELD OF THE INVENTION

The present invention relates to removal of hemoglobin from a sample. In particular the present invention relates to a method in which a protein with specificity towards hemoglobin and/or the complex of hemoglobin with haptoglobin is immobilized on a solid support and utilized to remove hemoglobin from a sample comprising hemoglobin, such as a hemolysed sample obtained from a subject.

### BACKGROUND OF THE INVENTION

The analysis of serum and plasma samples is a standard part of care for patients as well as a central part of research. However, serum and plasma samples can often be contaminated with excess hemoglobin resulting from hemolysis. Hemolysis is defined as the rupture of erythrocytes (i.e. red blood cells) with the release of hemoglobin and other cellular constituents into the plasma, or liquid portion of whole blood. The release of hemoglobin causes the serum or plasma to appear red in colour.

Hemolysis can be intravascular or extravascular. Intravascular hemolysis occurs very rarely and is usually the result of a transfusion reaction or hemolytic anemia. Extravascular hemolysis is quite common and means that the erythrocyte is lysed as part of an external process, usually in a clinical setting where the venepuncture process is used to obtain the sample. Extravascular hemolysis can also be caused by mechanical distortion, repeated freeze-thaw cycles, osmotic shock, or by excessive mixing of the sample.

The excess hemoglobin caused by hemolysis adversely affect the quantification of a range of clinical markers. It is not uncommon for physiological concentrations of free hemoglobin, such as below 0.005 g/L to be present in plasma. However, visible hemolysis start to appear at concentrations of ˜0.20 g/L and clearly visible hemolysis, such as 0.50 g/L may be the result of samples containing only 0.5% lysed erythrocytes. Many assays in standard clinical biochemistry laboratories relies on colourimetric measurements, with which the absorption of hemoglobin in the red-band area (around 350-550 nm) interferes to cause inaccurate results. Thus, even minute amounts of hemoglobin can complicate sample analysis tremendously.

In addition, hemoglobin has also been found to inhibit some enzymes and to interfere with other chemical methodologies.

Besides the interference with clinical assays for quantification of clinical markers, hemolysed samples may bias the quantification of analytes in other ways. Several constituents, such as potassium, are normally present in large amounts within the erythrocytes. When the erythrocytes burst these substances will be released and the measured parameters for the values will become falsely elevated in the surrounding serum. Also, when enough erythrocytes rupture and release their intracellular fluid into the surrounding extracellular area, as seen with gross hemolysis, the extracellular fluid becomes more dilute and analytes normally present in the extracellular fluid, such as sodium and chloride, will appear falsely low.

Overall, hemolysed samples accounts for 40-70% of all samples deemed unsuitable for analysis in accordance with the Total Allowable Error (TAE) limits by the College of American Pathologists (CAP) and Clinical Laboratory Improvement Amendments (CLIA) guidelines. Consequently, hemolysis is the leading cause of unsuitable samples by a large margin and significantly impacts and complicates the treatment of the patients and analysis of scientific data.

Amongst the analytes most sensitive to hemolysis are haptoglobin, lactate dehydrogenase (LDH), folate, aspartate transaminase (ASAT), transferrin, troponin T (TnT), lipase, paracetamol, gentamicin and ions such as potassium, ammonium, and phosphate. All of these analytes will either have their value elevated or lowered by 10% at a hemolysis index (H-index), with the H-index being a quantitative measure of the amount of hemoglobin (in mg/dL) in a sample. Visual examples of increasing H-index is given in FIG. 1.

The common methodology to systematically detect and reliably quantify hemolysis relies on an automated estimation of the H-index. Standardized procedures to determine if a serum/plasma sample is contaminated with hemoglobin already exist. If such an analysis is positive (i.e. H-index above a certain threshold), the laboratory may proceed by taking one of the following possible actions; i) report the sample result with a note of caution to alert the clinician ordering the sample, ii) reject the sample and prompt the clinician for a new sample, or iii) adjust the test result to by a predefined algorithm or procedure.

The first option is not satisfactory since certain parameters determined using the standard set-up are non-correct and to be ignored. The second option is not always feasible. Either the patient can suffer from a disease with increased hemolysis, the patient is not available for a novel blood sample immediately or the sample is of an older date and cannot be reproduced. Furthermore, re-sampling is both inconvenient and expensive. The third option is complicated and inaccurate because the breakdown of erythrocytes mainly depends on the distinct cell fragility and because the intracellular content of several molecules is widely variable among subjects, overall rendering the use of corrective procedures unreliable or even misrepresentative. Thus, neither of options i) through iii) are attractive solutions to handle hemolysed samples.

The overall picture is further worsened by the fact that little consensus currently appears to exist among different laboratories as to which of the above policies (i-iii) are to be adopted for hemolysed samples. Therefore, clinical results and decision-making based on a hemolysed sample may be affected by the H-index policy of the personnel group and/or laboratory handling the hemolysed sample.

As described herein, efforts have been put into defining guidelines for standardizing hemolysis detection by H-index, establishing H-index thresholds for sample rejection, and management of unreliable samples. On the other hand, relatively few solutions have been successfully implemented to remove the cause of the contaminated samples, mainly the excess of hemoglobin.

The traditional and widely accepted approach for reducing or removing hemoglobin from a sample is the red blood cell (RBC) membrane preparation process. This approach is based on consecutive steps of centrifugation and generally considered a highly reproducible method. However, the RBC membrane preparation process is very low capacity and labour-intensive method that is not suitable for high throughput processing of a plethora of samples. Previously, hemoglobin has been purified, separated or removed by functionalized solid phases. WO 2004/036189 demonstrated that hemoglobin binds to nickel, copper, zinc, or cobalt 3-[[[Bis(carboxymethyl)amino]-acetyl]amino]-propyl magnetic silica particles, and that these particles can be used to separate hemoglobin from proteins that do not bind to the particles. Similarly, Guo et at. (Applied Materials Interfaces (2016), 8, 29734-29741)) showed that hemoglobin from human whole blood could be isolated using mesoporous magnetic nanoparticles containing copper oxide.

Two products, HemogloBind and HemoVoid, for removal and capture of hemoglobin are commercially available. According to the product sheets supplied by the manufacturer, HemogloBind is based on a solid support to which is attached poly-electrolytes (polymers) that binds hemoglobin through for instance electrostatic interactions. Thus, upon usage, hemoglobin is bound to the matrix of HemogloBind, whereas the supernatant contains hemoglobin depleted serum or plasma. HemoVoid, is based on a silica-based protein enrichment matrix that binds hemoglobin through mixed-mode ligand combinations (ionic, hydrophobic, aromatic, polymer). Thus, HemoVoid may be used for depleting hemoglobin from a sample.

Common to the above functionalized solid phases for removal of hemoglobin is that they are all based on organic and inorganic functional moieties exerting a mixture of affinity interactions between hemoglobin and a solid phase matrix. These current methods utilizing functionalized solid phases are rather unspecific and may affect the measurements of other analytes due to non-specific absorption to and retention of other components on the solid phase matrix. In addition, it has been suggested that HemogloBind binds the complex of hemoglobin with haptoglobin less strongly, meaning that the capacity for hemoglobin removal is limited. Since a healthy person has a haptoglobin plasma concentration of 150 mg/dL and hemoglobin binds very strongly to haptoglobin, all hemoglobin up to an H-index of around 100 will be bound to haptoglobin.

Hence, an improved high throughput method for high-specificity removal of hemoglobin from a sample would be advantageous. In particular, a more efficient and/or reliable method for mass screening and processing of hemolysed samples in a clinical setting would be advantageous.

### SUMMARY OF THE INVENTION

The present invention relates to a method for removal of hemoglobin from a sample, such as a hemolysed sample. Preferably, only the amount of hemoglobin is lowered by the proposed method, thereby enabling the use of the technology as part of the standard procedure for handling blood samples in a clinical biochemical laboratory. A high level of selectivity is achieved by functionalizing a solid support with a non-mammalian protein which specifically binds hemoglobin and the prevalent complex of hemoglobin and haptoglobin. By incorporating the functionalized surface in the clinical standard procedure for detection of hemolysis, certain samples can be sent to hemoglobin removal, and the otherwise affected parameters afterwards determined on the treated samples.

Thus, an object of the present invention relates to the provision of an improved high throughput method for high-specificity removal of hemoglobin from a sample, such as a hemolysed sample.

In particular, it is an object of the present invention to provide a solid support functionalized by a non-mammalian protein which specifically binds hemoglobin and/or the complex of hemoglobin with haptoglobin, wherein the affinity and specificity of the functionalized solid support towards hemoglobin is improved as compared to known solid phase matrix based on organic or inorganic functional moieties.

Thus, one aspect of the invention relates to a method for removal of hemoglobin from a sample, the method comprising the following steps of:
a) providing a sample comprising hemoglobin
b) contacting the sample with at least one non-mammalian protein or protein fragment immobilized on a solid support, and
c) separating hemoglobin bound to the at least one non-mammalian protein or protein fragment from the sample,

thereby removing hemoglobin from the sample,

wherein said non-mammalian protein or protein fragment is derived from a unicellular organism and comprises a binding moiety which specifically binds hemoglobin and/or the complex of hemoglobin with haptoglobin.

Another aspect of the present invention is to provide a solid support to which is immobilized at least one non-mammalian protein or protein fragment derived from a unicellular organism and comprising a binding moiety, which specifically binds hemoglobin and/or the complex of hemoglobin with haptoglobin.

Yet another aspect of the present invention is to provide a container comprising a solid support as described herein.

Still another aspect of the present invention is to provide a kit for removal of hemoglobin from a sample, the kit comprising:
i. a container as described herein, and
ii. instructions for use.

A further aspect of the present invention relates to the use of a non-mammalian protein or protein fragment for removal of hemoglobin from a sample, wherein said non-mammalian protein or protein fragment is derived from a unicellular organism and comprises a binding moiety which specifically binds hemoglobin and/or the complex of hemoglobin with haptoglobin.

### BRIEF DESCRIPTION OF THE FIGURES

FIG. 1 shows test tubes with increasing degree of hemolysis/H-index (from left to right).

FIG. 2 shows examples of non-proteinogenic amino acids.

FIG. 3 shows an overview of the different IsdH constructs produced in example 1.

FIG. 4 shows an SDS-PAGE displaying bands and MW of the used IsdH constructs produced in example 1.

FIG. 5 shows (A) the efficiency of IsdH-C-Sepharose, Sham-Sepharose and Hemoglobind in hemoglobin removal compared to an untreated plasma sample. (B) Three column materials after incubation with 3 mg/ml hemoglobin plasma and centrifugation. From left to right the vials contain: Sham-Sepharose, Hemoglobind, IsdH-C-Sepharose.

FIG. 6 shows plasma parameters measured before and after addition of hemoglobin and/or exposure to the different Sepharose types.

FIG. 7 shows a plot of the measured plasma parameters with hemolysis normalized to same plasma without added hemolysate. To compare the different plasma parameters, the albumin concentration (measurement of which is not sensitive to hemolysis), was used for normalization of all samples, with plasma without hemolysate as reference.

FIG. 8 shows the effect on H-index, measured as hemoglobin in mg/dl after treatment of hemolyzed plasma with different type of Sepharose and HemogloBind.

FIG. 9 shows measured haptoglobin levels of hemolyzed plasmasamples after exposure to the different Sepharose types.

FIG. 10A-G shows measurements of a range of blood biochemical parameters (aspartate transaminase, bilirubin, iron, I-index, troponine, alkaline phosphatase and paracetamol) after hemoglobin was removed from the samples using the different Sepharoses.

FIG. 11A-D shows measurements of a range of parameters (calcium, potassium, thyroxin and thyrotropine) after hemoglobin was removed from the samples using the different Sepharoses.

FIG. 12 shows a clinical setting setup with (A) measured hemoglobin concentration subsequent to exposure of patient samples to either IsdH-C-Sepharose, Sham-Sepharose or Hemoglobind, and (B) the same data presented as percentage of initial hemoglobin remaining in the samples.

FIG. 13 shows a clinical setting setup with measured haptoglobin concentration subsequent to exposure of patient samples to either IsdH-C-Sepharose, Sham-Sepharose or Hemoglobind.

FIG. 14 shows measurement of a range of parameters (albumin, alkaline phosphatase, conjugated bilirubin, creatinine kinase, and ferritin) subsequent to exposure of patient samples to either IsdH-C-Sepharose, Sham-Sepharose or Hemoglobind. Measurements that were assigned out of range due to hemolysis are marked in grey.

FIG. 15 shows (A) the hemoglobin clearance capacity and (B) haptoglobin clearance capacity of Sepharoses with other non-mammalian proteins (IsdB, Htaa and Shr) attached thereto.

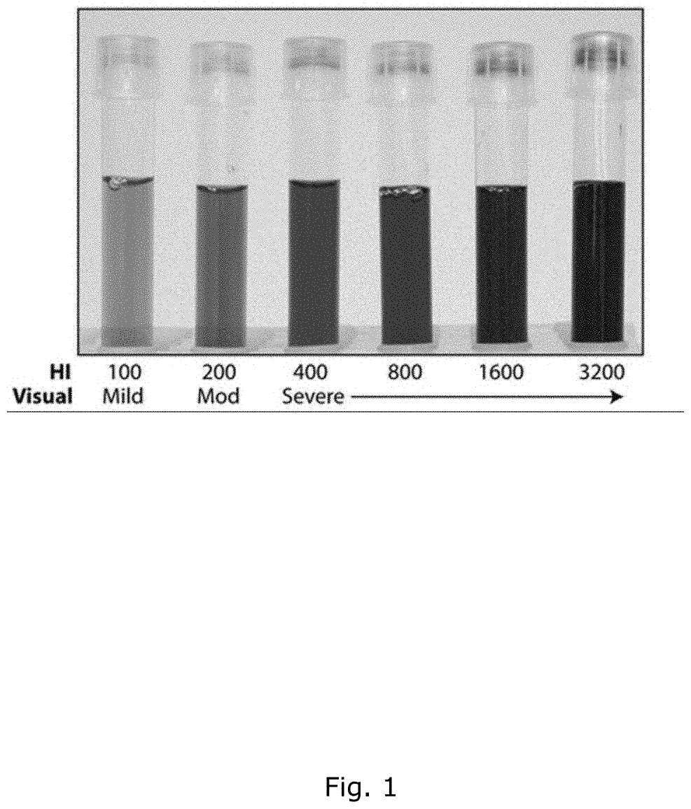

The present invention will be described in more detail in the following.

### DETAILED DESCRIPTION OF THE INVENTION

### Definitions

Prior to discussing the present invention in details, the following terms and conventions will first be defined:

Amino Acid Linker

In the present context, the term “amino acid linker” refers to a consecutive stretch of amino acid of any length, which is added to a protein to link the protein to another entity, thus functioning as a coupling moiety. The amino acid linker may for instance be used to couple a protein to a solid support.

Analyte

In the present context, the term “analyte” refers to any ion, molecule or biochemical entity that may be contained in a sample (e.g. plasma, serum, whole blood). The quantity of the analyte may be measured by any clinical biochemical unit in their standard test of the sample.

In the present context, the terms “analyte”, “clinical marker”, and “biochemical parameter” may be used interchangeably.

Binding Moiety

In the present context, the term “binding moiety” refers to a part of a protein that has specific affinity towards hemoglobin and/or the complex of hemoglobin with haptoglobin. The binding moiety may be any general class of binding domains known to bind hemoglobin and/or the complex of hemoglobin with haptoglobin (e.g. NEAT domains, CR domains etc.) or by a specific sequence of amino acids.

Consensus Sequence

In the present context, the term “consensus sequence” refers to any amino acid sequence introduced into a protein, which may serve as a site for crosslinking another molecule to the protein via an enzymatic catalysed coupling. A non-limiting example of an enzyme that can recognize a consensus sequence and facilitate crosslinking is transglutaminase.

Coupling Moiety

In the present context, the term “coupling moiety” refers to a part of a protein that may be used to attach the protein with another entity (e.g. a solid support, surface, bead, molecule, protein etc.). The coupling moiety represents a unique site within the protein.

The coupling moiety may be an integral part of the protein (e.g. introduced by a mutation/recombinantly) or be located in conjunction with the protein at either the N- or C-terminal end of the protein.

The coupling moiety can be any type of entity (e.g. amino acid residue(s), polymers, functional chemical groups) that may be used to couple the protein to a second entity and may be any length (e.g. a single amino acid residue or a longer stretch of consecutive amino acids)

Thus, in the present context, a coupling moiety may be, but is not limited to, a cysteine residue, an unnatural amino acid, an amino acid linker, an amino group, a consensus sequence and polyethylene glycol (PEG).

Hemolysis Index (H-Index)

In the present context, the term “hemolysis index (H-index)” refers to a measure of the concentration of hemoglobin within a sample. Thus, the conventional units for H-index is H=1 equals 1 mg/dl of hemoglobin (or 0.621 μmol/l in SI units).

In the present context, the difference in the H-index before and after treatment of a hemolysed sample constitutes a measure of the efficiency of hemoglobin removal. Thus, it is preferable to achieve a low H-index subsequent to treatment of the sample with the protein that has specific affinity towards hemoglobin and/or the complex of hemoglobin with haptoglobin.

The H-index can be measured by absorbance, preferably after using a modified version of Drapbkin's reagent converting all hemoglobin to cyanmethemoglobin, which has an absorbance maximum at 540 nm. The hemoglobin concentration in a sample can then be found by referring to a standard curve made from a hemoglobin sample with known concentration.

Immobilization

In the present context, the term “immobilization” refers to the attachment of a protein to another entity (e.g. a solid support, surface, bead, molecule, protein etc.).

The attachment of the protein with another entity is accomplished via interaction with a unique coupling moiety located within or in conjunction with the protein.

Immobilization may be of either covalent or non-covalent nature. Thus, covalent attachment of the protein to another entity may be achieved by an approach such as, but not limited to, an amino acid linker, conjugation via unnatural or cysteine amino acid residue(s) within the protein or enzymatic coupling via a consensus sequence within the protein. Additionally, immobilization may be achieved by a variety of interactions such as, but not limited to, hydrophobic interactions, hydrophilic interactions, ionic interactions, van der walls forces, hydrogen bonding, and combinations thereof.

Near Iron Transporter (NEAT) Domain

In the present context, the term “near iron transporter (NEAT) domain” refers to a group of iron-interactive protein domains found exclusively in bacteria. NEAT domains binds specifically hemoglobin and/or the complex of hemoglobin with haptoglobin.

NEAT domains may be encoded by gram-positive pathogens such as, but not limited to, Bacillus anthracis, Staphylococcus aureus, Streptococcus pyogenes, Clostridium perfringens and Listeria monocytogenes, or non-pathogens such as, but not limited to, Bacillus halodurans and Listeria innocua.

Non-Mammalian Protein

In the present context, the term “non-mammalian protein” refers to any protein not originating from a vertebrate within the class Mammalia.

Protein Fragment

In the present context, the term “protein fragment” refers to a part or subset of a full-length protein. Thus, the protein fragment consists of subset of the amino acids constituting the full-length protein.

Recombinant

In the present context, the term “recombinant” when referring to a protein, means that a protein is derived from recombinant (e.g. microbial or mammalian) expression systems.

Similarly, the term “recombinantly introduced” when referring to one or more residues of a protein, means that the one or more residues is introduced when expressing a protein recombinantly. This may for instance be the addition or substitution of a cysteine residue during recombinant expression.

Sample

In the present context, the term “sample” refers to a liquid specimen obtained from a subject. Preferably, the sample contains hemoglobin (e.g. plasma, serum, whole blood samples).

The sample may be obtained in a clinical setting with the aim of determining the presence and/or quantity of one or more analytes contained therein.

Solid Support

In the present context, the term “solid support” refers to any surface on which a protein can be attached or interact with. The solid support can any form (e.g. flat, spherical, elongated, cylindrical etc.), and be of any material (e.g. glass, plastic, Sepharose, dextran etc.).

A solid support may be comprised of a combination of materials. Furthermore, a solid support functionalized with a protein may be used to coat another surface (e.g. a test tube, bead, microtiter plate etc.).

In the present context, the terms “solid support”, “solid phase” and “matrix” are used interchangeably.

Sequence Identity

In the present context, the term “identity” is here defined as the sequence identity between genes or proteins at the nucleotide, base or amino acid level, respectively. Specifically, a DNA and a RNA sequence are considered identical if the transcript of the DNA sequence can be transcribed to the identical RNA sequence.

Thus, in the present context “sequence identity” is a measure of identity between proteins at the amino acid level and a measure of identity between nucleic acids at nucleotide level. The protein sequence identity may be determined by comparing the amino acid sequence in a given position in each sequence when the sequences are aligned. Similarly, the nucleic acid sequence identity may be determined by comparing the nucleotide sequence in a given position in each sequence when the sequences are aligned.

To determine the percent identity of two amino acid sequences or of two nucleic acids, the sequences are aligned for optimal comparison purposes (e.g., gaps may be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In one embodiment, the two sequences are the same length.

In another embodiment, the two sequences are of different length and gaps are seen as different positions. One may manually align the sequences and count the number of identical amino acids. Alternatively, alignment of two sequences for the determination of percent identity may be accomplished using a mathematical algorithm. Such an algorithm is incorporated into the NBLAST and XBLAST programs of (Altschul et al. 1990). BLAST nucleotide searches may be performed with the NBLAST program, score=100, wordlength=12, to obtain nucleotide sequences homologous to a nucleic acid molecules of the invention. BLAST protein searches may be performed with the XBLAST program, score=50, wordlength=to obtain amino acid sequences homologous to a protein molecule of the invention.

To obtain gapped alignments for comparison purposes, Gapped BLAST may be utilized. Alternatively, PSI-Blast may be used to perform an iterated search, which detects distant relationships between molecules. When utilising the NBLAST, XBLAST, and Gapped BLAST programs, the default parameters of the respective programs may be used. See http://www.ncbi.nlm.nih.gov. Alternatively, sequence identity may be calculated after the sequences have been aligned e.g. by the BLAST program in the EMBL database (www.ncbi.nlm.gov/cgi-bin/BLAST). Generally, the default settings with respect to e.g. “scoring matrix” and “gap penalty” may be used for alignment. In the context of the present invention, the BLASTN and PSI BLAST default settings may be advantageous.

The percent identity between two sequences may be determined using techniques similar to those described above, with or without allowing gaps. In calculating percent identity, only exact matches are counted. An embodiment of the present invention thus relates to sequences of the present invention that has some degree of sequence variation.

Unnatural Amino Acid

In the present context, the term “unnatural amino acid” refers to non-proteinogenic amino acids that either occur naturally or are chemically synthesized. Thus, unnatural amino acids encompass any amino acid that is not among the 20 encoded proteinogenic (or standard/natural/canonical) amino acids.

In the present context, the term “unnatural amino acid” also include non-standard amino acids or non-canonical amino acids.

Unnatural amino acids may contain functional groups such as, but not limited to, keto, acetylene, azide, and boronate, which may be used to selectively introduce a large number of biophysical probes, tags, and novel chemical functional groups into proteins in vitro or in vivo. Thus, unnatural amino acid may be used for conjugation of a protein to another entity via a tag or chemical functional group using a technique such as click-chemistry.

Unicellular Organism

In the present context, the term “unicellular organism” (or single cell organism) refers to any organism consisting of only one cell (e.g. bacteria, protozoa and unicellular fungi). The unicellular organism may be either prokaryotic or eukaryotic.

Method for Removing Hemoglobin from a Sample

Analysis of clinical markers can be affected by either endogenous or exogenous biochemical molecules. Endogenous interference occurs when biochemical molecules found naturally in the sample of a patient bias the sample result leading to inaccurate conclusions. One such endogenous biochemical molecule is hemoglobin, which is present in excess amounts in hemolysed samples. Hemolysis can occur in vivo, but the major problem that clinical laboratories get is that it occurs during and after collection of specimens. Thus, hemolysis accounts for approximately 40-70% of all samples deemed unsuitable for analysis. Consequently, laboratories need to systematically detect and reliably quantify hemolysis in every collected sample by means of objective and consistent technical tools that assess sample integrity. This is currently done by automated estimation of hemolysis index (H-index). However, lack of harmonization of the assessment of the H-index is an issue and little consensus currently appears to exist among different laboratories on how to handle samples characterized by a given H-index.

The present invention aims at providing means for removing the major cause of samples being deemed unsuitable for analysis. Thus, the present invention relates to a method for removal of hemoglobin from a sample, such as a hemolysed sample.

Thus, an aspect of the present invention relates to a method for removal of hemoglobin from a sample, the method comprising the following steps of:
a) providing a sample comprising hemoglobin
b) contacting the sample with at least one non-mammalian protein or protein fragment immobilized on a solid support, and
c) separating hemoglobin bound to the at least one non-mammalian protein or protein fragment from the sample,

thereby removing hemoglobin from the sample,

wherein said non-mammalian protein or protein fragment is derived from a unicellular organism and comprises a binding moiety which specifically binds hemoglobin and/or the complex of hemoglobin with haptoglobin.

By utilizing proteins with specific affinity towards hemoglobin and/or the complex of hemoglobin with haptoglobin, the amount of non-specific binding of non-target molecules is lowered compared to other similar techniques, such as techniques using solid phases functionalized with synthetic polymers. Thus, the method as described herein allows for the removal of hemoglobin without biasing other biochemical constituents of the sample and enables the use of the technology as part of the standard procedure for handling blood samples in a clinical biochemical laboratory. By incorporating the functionalized surface in the clinical standard procedure for detection of hemolysis, certain samples can be sent to hemoglobin removal, and the otherwise affected parameters afterwards determined on the treated samples.

Origin of the Non-Mammalian Protein

A high level of selectivity towards hemoglobin is achieved by functionalizing a solid support with a non-mammalian protein, which specifically binds hemoglobin and the prevalent complex of hemoglobin and haptoglobin.

Iron-harvesting proteins is found ubiquitously in nature, where iron is an important resource, e.g. as an enzymatic cofactor. As a result, most living organisms require iron to survive and replicate. A common source of iron is heme compounds that can sequester iron and organize its distribution and availability in the organism.

Many organisms that encompass heme compounds also comprise proteins that specifically interact with these compounds. Proteins that specifically bind heme compounds, such as hemoglobin, are suited for the removal of hemoglobin from hemolysed samples.

Thus an embodiment of the present invention relates to a method as described herein, wherein the unicellular organism is selected from the group consisting of bacteria, fungi and protozoa.

Another embodiment of the present invention relates to a method as described herein, wherein the non-mammalian protein or protein fragment is HpHbR (SEQ ID NO:9) from Trypanosoma.

A further embodiment of the present invention relates to a method as described herein, wherein the non-mammalian protein or protein fragment is Rbt51 (SEQ ID NO:10) from Candida.

Some of the most studied organisms in relation to iron acquisition are pathogenic bacteria. Iron deprivation hinders the attempts of pathogenic bacteria to colonize the human body, and bacteria that can use heme and hemoglobin can take advantage of a major iron-reservoir in their host environment. Consequently, mechanisms for iron acquisition from hemo-proteins are very common among bacterial pathogens. In order to obtain iron from the host, bacteria exploit hemoglobin by targeting heme stored within.

Therefore, an embodiment of the present invention relates to a method as described herein, wherein the unicellular organism is a pathogenic bacterium.

Non-mammalian proteins or protein fragments that may be utilized to work the invention can further originate from gram positive or gram negative bacteria.

Thus an embodiment of the present invention relates to a method as described herein, wherein the pathogenic bacterium is a gram positive or gram negative bacteria.

The mechanism which these two groups of bacteria apply for interaction with heme or hemoglobin differs. A prevalent interaction in gram negative bacteria is the specific binding of hemo-proteins by the TonB-dependent outer membrane receptor. The heme is subsequently transposed over the inner membrane by an ABC transporter complex.

TonB-dependent transporters are membrane proteins that bind and transport ferric chelates called siderophores. These transporters show high affinity and specificity for siderophores and require energy derived from the proton motive force across the inner membrane to transport them. The energy force is provided through interaction with an inner membrane protein complex consisting of TonB, ExbB, and ExbD.

In addition to the TonB-dependent specific binding of heme and hemo-proteins, other proteins derived from gram negative bacteria have been shown to interact with hemoglobin or complexes of hemoglobin. In Yersenia enterocolitica the protein HemR has been identified, in Haemophilus influenza the protein-triplet of HgpA, HgpB and HgpC have been identified, and in Neisseria meningitides the protein HpuB has been identified.

Thus, an embodiment of the present invention relates to a method as described herein, wherein the gram negative bacteria is selected from the group consisting of Y. enterocolitica, H. influenza and N. meningitidis.

Another embodiment of the present invention relates to a method as described herein, wherein the non-mammalian protein or protein fragment is selected from the group consisting of HemR (SEQ ID NO:11), HgpA (SEQ ID NO:12), HgpB (SEQ ID NO:13), HgpC (SEQ ID NO:14) and HpuB (SEQ ID NO:15).

The heme acquisition mechanism of gram negative bacteria is most likely not utilized by gram positive bacteria which have a different cell membrane organization and therefore handle heme and hemo-protein by alternative mechanisms.

Many gram positive bacteria acquire iron through the interaction of bacterial proteins with heme or hemoglobin. Proteins with affinity towards hemoglobin in general comprise one or more binding domains (or moieties) which are mostly responsible for the specific interaction with hemoglobin. Binding domains has been shown to be largely conserved across a large variety of gram positive bacteria.

Thus, an embodiment of the present invention relates to a method as described herein, wherein the gram positive bacterium is of the phylum firmicutes.

Another embodiment of the present invention relates to a method as described herein, wherein the firmicute is of a genus selected from the group consisting of Bacillus ssp., Staphylococcus ssp., Streptococcus ssp., Clostridium ssp. and Listeria ssp.

Binding domains with specific affinity towards hemoglobin and/or the complex of hemoglobin with haptoglobin The rate of binding is called affinity, and this measurement typifies a tendency or strength of the effect. The binding domain (or moiety) of the non-mammalian protein or protein fragment has high affinity towards hemoglobin and/or the complex of hemoglobin with haptoglobin.

The binding of hemoglobin to the binding domain may occur by intermolecular forces, such as ionic interactions, hydrogen bonds and Van der Waals forces. Binding of hemoglobin to the binding domain may also be affected by the chemical conformation or three-dimensional shape of the binding domain.

Binding domains with affinity towards hemoglobin, such as the near iron transporter (NEAT) and the conserved region (CR) domain, have been identified in several gram positive bacteria. Therefore, an embodiment of the present invention relates to a method as described herein, wherein the gram positive bacteria is selected from the group consisting of S. aureus, S. pyrogenes and C. diphteriae.

Another embodiment of the present invention relates to a method as described herein, wherein the binding moiety comprises at least one CR domain.

C. diphtheriae utilizes hemoglobin as iron sources for growth in iron depleted environments. The use of hemin-iron in C. diphtheriae involves the dtxR- and iron-regulated hmu hemin uptake locus, which encodes an ABC hemin transporter, and the surface anchored hemin binding proteins HtaA and HtaB. The HtaA protein comprises a CR domain that may be divided into the two 150 amino acid domains CR1 and CR2, both of which are able to bind hemoglobin.

Therefore, an embodiment of the present invention relates to a method as described herein, wherein the non-mammalian protein or protein fragment is HtaA (SEQ ID NO:16) or a HtaA homologue.

Another embodiment of the present invention relates to a method as described herein, wherein the binding moiety comprises at least one domain selected from the group consisting of CR1 (SEQ ID NO:17), CR2 (SEQ ID NO:18), and combination thereof.

NEAT domains are predicted to contain a β-strand secondary structure, and are typically found in proteins anchored to the cell membrane or cell wall of gram positive bacteria. The NEAT-containing proteins expressed by pathogenic bacteria function together to scavenge heme from host hemo-proteins, such as hemoglobin, and transfer it to and through the cell surface for delivery into the bacterial cytosol where iron is released.

Therefore, an embodiment of the present invention relates to a method as described herein, wherein the binding moiety comprises at least one near iron transporter (NEAT) domain.

The heme binding function of the NEAT domains is conserved across a large pool of gram positive bacteria. NEAT domains can bind heme and/or hemoglobin, extract heme from hemoglobin by a physical interaction, and undergo NEAT-NEAT heme transfer events. These functions are based on conserved, specific secondary structural regions of the NEAT domain, as well as specific amino acids within the heme-binding pocket. NEAT domains are composed of eight β-strands and a small 3₁₀-helix that fold to form a heme-binding pocket, both which encompass some amino acid motifs that are conserved through NEAT domains.

Thus, an embodiment of the present invention relates to a method as described herein, wherein the at least one near iron transporter (NEAT) domain comprises a amino acid motif selected from the group consisting of SXXXXY, YXXXY, and the combination thereof. Here X represents any amino acid.

Hemolysis is a complication in septic infections with Staphylococcus aureus, which utilizes the released hemoglobin as an iron source. S. aureus secretes an α-hemolysin that integrates in red blood cell membranes and induces osmotic hemolysis. Liberation of hemoglobin into plasma facilitates S. aureus acquisition of iron by means of an iron-sequestering pathway designated the iron-regulated surface determinant (Isd) system. Extraction of heme is achieved by the two bacterial surface-exposed hemoglobin-receptors of the Isd family, namely IsdB (SEQ ID NO:7) and IsdH (SEQ ID NO:8). The heme-binding function of IsdB and IsdH is conferred by the presence of a NEAT domain. Thus, IsdH and IsdB constitute potential candidates for proteins that may be immobilized on a solid support and utilized for the removal of hemoglobin from a sample.

Thus, an embodiment of the present invention relates to a method as described herein, wherein the NEAT domain comprises:
i. SEQ ID NO:7 or a NEAT domain having at least 90% sequence identity to the full-length sequence of SEQ ID NO:7, or
ii. SEQ ID NO:8 or a NEAT domain having at least 90% sequence identity to the full-length sequence of SEQ ID NO:8.

Another embodiment of the present invention relates to a method as described herein, wherein the NEAT domain comprises:
i. SEQ ID NO:7 or a NEAT domain having at least 90% sequence identity to the full-length sequence of SEQ ID NO:7, or
ii. SEQ ID NO:8 or a NEAT domain having at least 90% sequence identity to the full-length sequence of SEQ ID NO:8.

A further embodiment of the present invention relates to a method as described herein, wherein the NEAT domain is selected from the group consisting of:
i. SEQ ID NO:7 or a NEAT domain having at least 75%, such as at least 80%, such as at least 85%, such as at least 90%, such as at least 95%, such as at least 99%, sequence identity to the full-length sequence of SEQ ID NO:7, or
ii. SEQ ID NO:8 or a NEAT domain having at least 75%, such as at least 80%, such as at least 85%, such as at least 90%, such as at least 95%, such as at least 99%, sequence identity to the full-length sequence of SEQ ID NO:8.

Yet another embodiment of the present invention relates to a method as described herein, wherein the NEAT domain comprises SEQ ID NO:8 or a NEAT domain having at least 75%, such as at least 80%, such as at least 85%, such as at least 90%, such as at least 95%, such as at least 99%, sequence identity to the full-length sequence of SEQ ID NO:8.

The affinity of IsdH towards hemoglobin and/or the complex of hemoglobin with haptoglobin may be increased by genetic engineering of the protein. Thus, single substitution/mutations of amino acids, such as Y to A, can enhance the efficiency of hemoglobin removal from a sample. In one instance a Y642A mutation was introduced into IsdH (SEQ ID NO:19). The position of the mutation (i.e. residue 642) is in relation to the native IsdH protein.

Therefore, an embodiment of the present invention relates to the method as described herein, wherein the NEAT domain comprises SEQ ID NO:19.

IsdH contains three NEAT domains of which the first and second NEAT domain (IsdH^{N1} (SEQ ID NO:1) and IsdH^{N2} (SEQ ID NO:2)) bind to hemoglobin but lack heme binding activity, whereas the third, C-terminal, NEAT domain (IsdH^{N3} (SEQ ID NO:3)) carries the single heme-binding site of IsdH. The IsdH^{N3} domain may comprise a Y642A mutation (SEQ ID NO:20). IsdH^{N2} and IsdH^{N3} are connected by an α-helical linker domain. IsdB has a two-NEAT domain (IsdB^{N1} (SEQ ID NO:4) and IsdB^{N2} (SEQ ID NO:5)) structure connected with an α-helical linker domain, similar to the one of IsdH. Furthermore, the protein Shr (SEQ ID NO:6) from Streptococcus pyogenes also comprise two NEAT domains with affinity towards hemoglobin. The NEAT domains of Shr are denoted Shr^{N1} (SEQ ID NO:21) and Shr^{N2} (SEQ ID NO:22).

Therefore, an embodiment of the present invention relates to a method as described herein, wherein the at least one NEAT domain comprises a sequence selected from the group consisting of:
i. SEQ ID NO:1, SEQ ID NO:2, SEQ ID NO:3, SEQ ID NO:4, SEQ ID NO:5, SEQ ID NO:6, and combinations thereof, or
ii. a NEAT domain having at least 75% sequence identity to the full-length sequences of any one of SEQ ID NOs:1-6 and combinations thereof.

Another embodiment of the present invention relates to a method as described herein, wherein the NEAT domain has at least 90% sequence identity to the full-length sequences of any one of SEQ ID NOs:1-6 and combinations thereof.

Yet another embodiment of the present invention relates to a method as described herein, wherein the at least one NEAT domain comprises a sequence selected from the group consisting of:
i. SEQ ID NO:1, SEQ ID NO:2, SEQ ID NO:3, SEQ ID NO:4, SEQ ID NO:5, SEQ ID NO:6, and combinations thereof, or
ii. a NEAT domain having at least 75%, such as at least 80%, such as at least 85%, such as at least 90%, such as at least 95%, such as at least 99%, sequence identity to the full-length sequences of any one of SEQ ID NOs:1-6 and combinations thereof.

A further embodiment of the present invention relates to a method as described herein, wherein the at least one NEAT domain comprises a sequence selected from the group consisting of:
i. SEQ ID NO:1, SEQ ID NO:2, SEQ ID NO:3, SEQ ID NO:4, SEQ ID NO:5, SEQ ID NO:20, SEQ ID NO:21, SEQ ID NO:22, and combinations thereof, or
ii. a NEAT domain having at least 75% sequence identity to the full-length sequences of any one of SEQ ID NOs:1-5, SEQ ID Nos:20-22 and combinations thereof.

A still further embodiment of the present invention relates to a method as described herein, wherein the NEAT domain has at least 90% sequence identity to the full-length sequences of any one of SEQ ID NOs:1-5, SEQ ID Nos:20-22 and combinations thereof.

An even further embodiment of the present invention relates to a method as described herein, wherein the at least one NEAT domain comprises a sequence selected from the group consisting of:
i. SEQ ID NO:1, SEQ ID NO:2, SEQ ID NO:3, SEQ ID NO:4, SEQ ID NO:5, SEQ ID NO:20, SEQ ID NO:21, SEQ ID NO:22, and combinations thereof, or
ii. a NEAT domain having at least 75%, such as at least 80%, such as at least 85%, such as at least 90%, such as at least 95%, such as at least 99%, sequence identity to the full-length sequences of any one of SEQ ID NOs:1-5, SEQ ID Nos:20-22 and combinations thereof.

Hemoglobin released into human plasma during hemolysis binds rapidly to plasma haptoglobin and it is therefore preferred that the non-mammalian protein or protein fragment has affinity towards not only hemoglobin, but also the complex hemoglobin with haptoglobin. The inventors have shown that IsdH only binds the complex hemoglobin with haptoglobin via a direct hemoglobin interaction without direct contact to the haptoglobin subunit. Thus, fragments of IsdH as described above constitute suitable candidates for protein fragments that may be immobilized on a solid support and utilized for the removal of hemoglobin from a sample.

Consequently, an embodiment of the present invention relates to a method as described herein, wherein the NEAT domain comprises a sequence selected from the group consisting of:
i. SEQ ID NO:1, SEQ ID NO:2, SEQ ID NO:3, and combinations thereof, or
ii. a NEAT domain having at least 75% sequence identity to the full-length sequence of any one of SEQ ID NOs:1-3 and combinations thereof.

Another embodiment of the present invention relates to a method as described herein, wherein the NEAT domain has at least 90% sequence identity to the full-length sequence of any one of SEQ ID NOs:1-3 and combinations thereof.

Yet another embodiment of the present invention relates to a method as described herein, wherein the NEAT domain comprises:
i. SEQ ID NO:2, or
ii. a NEAT domain having at least 90% sequence identity to the full-length sequence of SEQ ID NO:2.

A further embodiment of the present invention relates to a method as described herein, wherein the NEAT domain comprises:
i. SEQ ID NO:1 or a NEAT domain having at least 90% sequence identity to the full-length sequence SEQ ID NO:1, and
ii. SEQ ID NO:2 or a NEAT domain having at least 90% sequence identity to the full-length sequence SEQ ID NO:2.

A still further embodiment of the present invention relates to a method as described herein, wherein the NEAT domain consists of:
i. SEQ ID NO:1 or a NEAT domain having at least 90% sequence identity to the full-length sequence SEQ ID NO:1, and
ii. SEQ ID NO:2, or a NEAT domain having at least 90% sequence identity to the full-length sequence SEQ ID NO:2.

An even further embodiment of the present invention relates to a method as described herein, wherein the NEAT domain is selected from the group consisting of:
i. SEQ ID NO:1, SEQ ID NO:2, SEQ ID NO:3, and combinations thereof, or
ii. a NEAT domain having at least 90% sequence identity to the full-length sequence of any one of SEQ ID NOs:1-3 and combinations thereof.

Immobilization of Protein on Solid Support

For efficient removal of hemoglobin from a sample, the non-mammalian protein or protein fragment with affinity towards hemoglobin and/or the complex of hemoglobin with haptoglobin is immobilized on a solid support.

Immobilization may be achieved by either covalent or non-covalent interactions, including, but not limited to, interactions such as hydrophobic interactions, hydrophilic interactions, ionic interactions, van der walls forces, hydrogen bonding, and combinations thereof.

Preferably, the immobilization is mediated by a coupling moiety, which constitutes a unique site within or at the ends of the protein or protein fragment. Thus, a preferred embodiment of the present invention relates to a method as described herein, wherein the at least one non-mammalian protein or protein fragment comprises a coupling moiety.

Another embodiment of the present invention relates to a method as described herein, wherein the at least one non-mammalian protein or protein fragment is immobilized on the solid support via the coupling moiety.

Many strategies exist for coupling proteins to surfaces and for the purpose of the present invention, any such strategy may be applied. Thus, the coupling may be performed by techniques such as, but not limited to, click chemistry, sulfhydryl chemistry, enzymatic coupling or polymeric linkers.

Methods exist for incorporating click reaction partners onto and into biomolecules, including the incorporation of unnatural amino acids containing reactive groups into proteins and the modification of nucleotides. Click chemistry does not refer to a single specific reaction, but describes a way of generating products that follow examples in nature, which also generates substances by joining small modular units. Click reactions occur in one pot, are not disturbed by water, and generate minimal and inoffensive byproducts.

Examples of click chemistry reactions include, but are not limited to, copper(I)-catalyzed azide-alkyne cycloaddition (CuAAC), strain-promoted azide-alkyne cycloaddition (SPAAC) and strain-promoted alkyne-nitrone cycloaddition (SPANC).

Click chemistry may be used to incorporate unnatural amino acids (UAA) into the non-mammalian protein or protein fragment of the present invention. As an example, an UAA with an azide side group provides convenient access for cycloalkynes to proteins tagged with azidohomoalanine (AHA), an UAA.

Additionally it is possible to introduce UAAs in proteins recombinantly. This may for instance be accomplished by a technique wherein a tRNA charged with the UAA of interest is engineered to recognize a stop codon, which then adds the UAA in the growing polypeptide chain by a mechanism commonly referred to as nonsense codon suppression. The most commonly used nonsense codon is the amber or TAG codon.

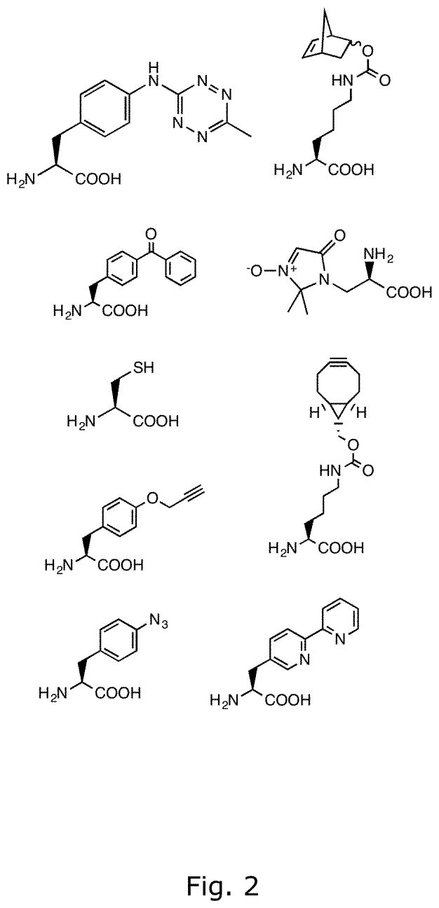

Examples of UAAs (e.g. non-proteinogenic amino acids) are displayed in FIG. 2.

One advantage of the incorporation of UAAs into the non-mammalian protein or protein fragment of the present invention is that coupling of the protein to the solid support may be conducted without consideration to the remaining amino acid sequence of the protein.

Sulfhydryl chemistry based on free cysteine residues located within the protein is another option for conjugation of the protein to a surface. Due to the rare accessibility of free cysteines at the protein surface, this amino acid residue does in many instances constitute a unique site in the protein that may be used as a selective coupling moiety. If no cysteine residue is present in the protein of interest, it may be introduced recombinantly to generate a unique site within the protein.

Coupling of proteins to surfaces may also be achieved by incorporation of consensus sequences into the protein. A protein may comprise one or more consensus sequences. A consensus sequences may serve as a site for crosslinking another molecule to the protein via an enzymatic catalysed coupling. A non-limiting example of an enzyme that can recognize a consensus sequence and facilitate crosslinking is transglutaminase.

Thus, an embodiment of the present invention relates to a method as described herein, wherein the coupling moiety is selected from a cysteine residue, an unnatural amino acid, an amino acid linker, an amino group, a consensus sequence and polyethylene glycol (PEG).

It is desirable to maximize the surface area and number of hemoglobin binding proteins relative to the solid support on which they are immobilized. Some polymeric structures have the ability to attach a large number of proteins at the distal ends of the polymer. Thus, an embodiment of the present invention relates to a method as described herein, wherein the coupling moiety is a dendrimeric structure or brush polymer.

Recombinant introduction of the coupling moiety into the protein ensures the uniqueness of the site of coupling, is a versatile strategy that may be employed to most proteins, and minimize perturbation of the protein structure by excluding involvement of “native” amino acid residues in the coupling. Thus, an embodiment of the present invention relates to a method as described herein, wherein the coupling moiety is recombinantly introduced into the at least one non-mammalian protein or protein fragment.

Another embodiment of the present invention relates to a method as described herein, wherein the coupling moiety is a cysteine residue recombinantly introduced into the at least one non-mammalian protein or protein fragment.

A further embodiment of the present invention relates to a method as described herein, wherein the non-mammalian protein comprises a sequence selected from the group consisting of:
i. SEQ ID NO:23, or
ii. a sequence having at least 75% sequence identity to the full-length sequence of SEQ ID NO:23, such as at least 80% sequence identity, such as at least 90% sequence identity.

Yet another embodiment of the present invention relates to a solid support, as described herein, to which is immobilized at least one non-mammalian protein or protein fragment derived from a unicellular organism and comprising a binding moiety, which specifically binds hemoglobin and/or the complex of hemoglobin with haptoglobin, wherein the non-mammalian protein comprises a sequence selected from the group consisting of:
i. SEQ ID NO:23, or
ii. a sequence having at least 75% sequence identity to the full-length sequence of SEQ ID NO:23, such as at least 80% sequence identity, such as at least 90% sequence identity.

An even further embodiment of the present invention relates to a method as described herein, wherein the non-mammalian protein comprises a sequence selected from the group consisting of:
i. SEQ ID NO:24, or
ii. a sequence having at least 75% sequence identity to the full-length sequence of SEQ ID NO:24, such as at least 80% sequence identity, such as at least 90% sequence identity.

Another embodiment of the present invention relates to a solid support, as described herein, to which is immobilized at least one non-mammalian protein or protein fragment derived from a unicellular organism and comprising a binding moiety, which specifically binds hemoglobin and/or the complex of hemoglobin with haptoglobin, wherein the non-mammalian protein comprises a sequence selected from the group consisting of:
i. SEQ ID NO:24, or
ii. a sequence having at least 75% sequence identity to the full-length sequence of SEQ ID NO:24, such as at least 80% sequence identity, such as at least 90% sequence identity.

For purposes of production of the non-mammalian protein or protein fragment, it is preferable to include in a recombinant protein a tag that facilitates the purification of the protein. A His-tag (or polyhistidine-tag) can be included at the N- or C-terminus of a protein, optionally followed by an amino acid sequence that enables the removal of the His-tag using endopeptidases. Alternatively, exopeptidases may be used to remove N-terminal His-tags subsequent to expression and purification.

Therefore, an embodiment of the present invention relates to a method as described herein, wherein the at least one non-mammalian protein or protein fragment further comprises a His-tag.

The solid support The non-mammalian protein or protein fragment may be immobilized on a range of solid supports consisting of a variety of materials. The material may be selected to complement the choice of form of the solid support. Thus, an embodiment of the present invention relates to a method as described herein, wherein the solid support is of a material selected from the group consisting of plastic, glass, metal, silica, polymers, Sepharose, dextran, carboxymethyl dextran, and combinations thereof.

The solid support may also be agarose.

For some applications, it may be desirable to coat a secondary surface (or container) with the solid support functionalized with an immobilized protein or protein fragment. If the secondary surface is a container, such as a vial, of either glass or plastic, a solid support of a material amenable for surface coating of such a container may be chosen. Thus, an embodiment of the present invention relates to a method as described herein, wherein the solid support is of carboxymethyl dextran.

The solid support of the present invention is not limited to any particular form (or shape) and can be provided in a form suitable for any type of assay. Preferred types of techniques wherein the functionalized solid support is utilized includes, but are not limited to, standard blood sampling, chromatography, microplate assays, and magnetic separation. Thus, an embodiment of the present invention relates to a method as described herein, wherein the solid support is in a form selected from the group consisting of a test tube, a resin, a microtiter plate, and a bead.

A preferred use is for the removal of hemoglobin from hemolysed samples in a clinical setting. Typically, samples in such a clinical setting is provided in a test tube. The test tube may be made of plastic or glass. Thus, an embodiment of the present invention relates to a method as described herein, wherein the solid support is a test tube. Another embodiment of the present invention relates to a method as described herein, wherein the test tube is made of plastic.

It is an ambition of the present invention that the method described herein may be directly implemented in the standard routine and handling of hemolysed samples. Thus, the method can be embedded directly with existing routines utilizing test tube for collecting blood samples from patients. Therefore, an embodiment of the present invention relates to a method as described herein, wherein the test tube is a blood collection tube made of plastic.

The blood collection tube may also be of another material, such as glass. Furthermore, if the solid support is in the form of a test tube, it may function as an insert in a blood collection tube or as a secondary vial into which the hemolysed sample is transferred if an H-index above a certain threshold is detected.

Sample Containing Hemoglobin

The method described herein may be used to specifically bind hemoglobin in any type of sample containing hemoglobin. A preferred use of the method is for the removal of hemoglobin from blood samples. Samples may thus comprise any fraction or constituents normally present in blood withdrawn from a patient. The sample may be pre-treated to removal of hemoglobin, e.g. to divide the sample into different fractions such as, but not limited to, serum, plasma and red cell lysates.

Thus, a preferred embodiment of the present invention relates to a method as described herein, wherein the sample is selected from the group consisting of whole blood, serum, plasma, and red cell lysates.

The samples may be from any subject, such as a human or animal subject.

Separation of Hemoglobin from the Remaining Sample

After contacting the sample with the immobilized protein, hemoglobin is bound to the functionalized solid support. Hemoglobin may be removed (or separated) from the initial sample by any known and standard technique. Depending on the form of the solid support, a suitable method of separation may be selected. Thus, for solid supports in the form of a resin, the method of separation could be elution or washing, and for solid supports in the form of beads the method of separation could be by magnetism (e.g. for magnetic beads).

Therefore, an embodiment of the present invention relates to a method as described herein, wherein the separation is accomplished by a technique selected from the group consisting of washing, eluting, filtration, centrifugation, magnetic separation, and combinations thereof.

Another embodiment of the present invention relates to a method as described herein, wherein the separation is accomplished by a technique selected from centrifugation or magnetic separation.

For solid supports in the form of test tube, the non-mammalian protein or protein fragment may be immobilized on the surface of the test tube, such as on the inner surface of the test tube. An embodiment of the present invention relates to a method as described herein, wherein the separation is accomplished by pipetting. Here the hemoglobin and/or the complex of hemoglobin with haptoglobin is bound to the non-mammalian protein or protein fragment immobilized on the surface of the test tube, and the remaining sample with reduced hemoglobin content may be removed by pipetting. Thus, an embodiment of the present invention relates to the method as described herein, wherein the non-mammalian protein or protein fragment is immobilized on a surface of the test tube and the separation is accomplished by pipetting.

Influence of Hemolysis on Analysis of Clinical Analytes

The method as described herein provides the means for removal of hemoglobin from a sample comprising excess amounts of hemoglobin, such as a hemolysed sample. However, hemolysis influence analysis of clinical analytes through different effects including, but not limited to, increased levels of hemoglobin. Thus, two effects also affecting the measurement of clinical analytes in the sample, but not related to the content of hemoglobin, are (i) dilution of the sample due to the volume addition from the lysed erythrocytes and (ii) elevated concentration of clinical analytes that are present in large amounts in erythrocytes. The affected clinical analytes are mainly ions. Thus, sodium and chloride will appear falsely low according to (i), whereas potassium is falsely elevated due to (ii). These effects cannot be mitigated by the method described herein and analytes affected by either (i) or (ii) will be biased.

Most other analyses of analytes that are directly affected by the presence of excess hemoglobin will benefit from the method as described herein. Especially the result of analytes which are quantified by colourimetric (or spectrophotometric) techniques will be improved and rescue the data from being inaccurate and to be ignored.

The threshold at which a clinical analyte is biased may be described by the H-index (i.e. the amount of hemoglobin in the sample). Thus, an analyte with a low H-index threshold is an indication that the analysis of the analyte is very sensitive to hemolysis, whereas a higher H-index indicates that the analysis of the analyte is less sensitive to hemolysis. Typically, the H-index threshold of clinical analytes varies from as high as H-index ˜600 (e.g. cholesterol, gamma glutamyltransferase (GGT), vancomycin) and above to as low as H-index 40 (e.g. haptoglobin, aspartate transaminase (ASAT), acetaminophen).

Thus, an embodiment of the present invention relates to a method as described herein, wherein the concentration of hemoglobin in the sample after step c) as described herein is reduced to at most 200 mg/dl, such as at most 150 mg/dl, such as at most 125 mg/dl, such as at most 100 mg/dl, such as at most 75 mg/dl, such as at most 50 mg/dl, such as at most 25 mg/dl.

Another embodiment of the present invention relates to a method as described herein, wherein the concentration of hemoglobin in the sample is reduced to at most 100 mg/dl.

A preferred embodiment of the present invention relates to a method as described, wherein the concentration of hemoglobin in the sample is reduced to at most 25 mg/dl.

The clinical analytes for which the analysis is rescued from being inaccurate by use of the method described herein includes, but are not limited to, aspartate transaminase (ASAT), troponin T (TnT), troponin I, creatinine, creatine kinase (CK or CPK), bilirubin (neonatal), bilirubin (total), cortisol, homocysteine, iron, transferrin, lipase, prostate specific antigen, testosterone, paracetamol, vitamine B12, parathyrine (PTH), γ-glutamyltransferase, gastrin, antitrypsin, alkaline phosphatase (ALKP), gentamicin, urate, acetaminophen, digoxin, valproic acid and vancomycin.

Thus, an embodiment of the present invention relates to a method as described herein, wherein the H-index of the sample is lowered to a level below the H-index threshold of the analyte of interest. Therefore, for some clinical analytes it may be sufficient to lower the H-index to 200, whereas for other clinical analytes it is necessary to lower the H-index to 25.

Solid Support or Container Comprising Protein which Specifically Binds Hemoglobin

The present invention also relates to a solid support whereto is immobilized a non-mammalian protein or protein fragment comprising a binding moiety with specific affinity towards hemoglobin and/or the complex of hemoglobin with haptoglobin. The solid support may be used for removal of hemoglobin from samples with excess hemoglobin.

Therefore, an aspect of the present invention relates to the provision of a solid support to which is immobilized at least one non-mammalian protein or protein fragment derived from a unicellular organism and comprising a binding moiety, which specifically binds hemoglobin and/or the complex of hemoglobin with haptoglobin.

The solid support can be used separately or as part of another entity. The solid support may for instance be coated onto another solid support such as, but not limited to, a test tube or a microtiter plate. For applications where the solid support is coated onto another entity (e.g. a surface of a vial, test tube, microtiter plate), the material of the solid support can be chosen to enable coating of the other entity.

An embodiment of the present invention relates to a solid support as described herein, wherein the material of the solid support is carboxymethyl dextran and the solid support is coated on another solid support or surface, such as a test tube or microtiter plate.

Another aspect of the present invention relates to the provision of a container comprising a solid support as described herein.

An embodiment of the present invention relates to a container as described herein, wherein the surface of the container is coated with the solid support.

A container coated with a solid support comprising an immobilized non-mammalian protein or protein fragment may take any form. Preferred uses of such a coated container are for the removal of hemoglobin in blood samples.

Thus, an embodiment of the present invention relates to a container as described herein, wherein the container is a test tube.

Another embodiment of the present invention relates to a container as described herein, wherein the container is made of plastic.

Yet another embodiment of the present invention relates to a container as described herein, wherein the container is made of glass.

A further embodiment of the present invention relates to a container as described herein, wherein the container is a blood collection tube made of plastic.

The coated container may be tailored to suit any existing protocol for handling of blood samples, including hemolysed samples. Thus, the coated container may be designed to fit into the workflow of a system for automated handling of blood samples. The coated container may be the primary container for storage of the blood sample or may be an insert that is detachably connected with the primary container for the blood sample. The container may also be a part of a protocol for handling blood samples, wherein the blood sample is transferred from one container to another.

The container may be part of a ready-to-use kit, which a physician can use immediately to remove hemoglobin from a sample. Such a kit may comprise one or more containers, such as an entire rack of containers that can be inserted into the workflow of a system for automated handling of blood samples.

Therefore, another aspect of the present invention relates to the provision of a kit for removal of hemoglobin from a sample, the kit comprising:
i. a container as described herein, and
ii. instructions for use.

Use of Non-Mammalian Protein for Removal of Hemoglobin from a Sample

The non-mammalian protein or protein fragment comprising a binding moiety, which specifically binds hemoglobin and/or the complex of hemoglobin with haptoglobin may be used alone for removal of hemoglobin from a sample.

Thus, another aspect of the present invention relates to the use of a non-mammalian protein or protein fragment for removal of hemoglobin from a sample, wherein said non-mammalian protein or protein fragment is derived from a unicellular organism and comprises a binding moiety, which specifically binds hemoglobin and/or the complex of hemoglobin with haptoglobin.

The non-mammalian protein or protein fragment may be expressed with a handle that enables physical separation of hemoglobin and/or the complex of hemoglobin with haptoglobin bound to the protein. The handle could be a tag, such as H6, which can be bound to moiety with affinity towards the tag, such as NiNTA. Alternatively, the handle could be biotin (or streptavidin) and the moiety with affinity towards the tag could be streptavidin (or biotin).

An alternative use of the present method is for the purification of hemoglobin. Purified hemoglobin may for instance be for used in a clinical setting or for scientific experiments.

It should be noted that embodiments and features described in the context of one of the aspects of the present invention also apply to the other aspects of the invention. Embodiments and features of the present invention are also outlined in the following items.

Items
1. A method for removal of hemoglobin from a sample, the method comprising the following steps of:
   a) providing a sample comprising hemoglobin
   b) contacting the sample with at least one non-mammalian protein or protein fragment immobilized on a solid support, and
   c) separating hemoglobin bound to the at least one non-mammalian protein or protein fragment from the sample,
   thereby removing hemoglobin from the sample,
   wherein said non-mammalian protein or protein fragment is derived from a unicellular organism and comprises a binding moiety which specifically binds hemoglobin and/or the complex of hemoglobin with haptoglobin.
2. The method according to item 1, wherein the unicellular organism is selected from the group consisting of bacteria, fungi and protozoa.
3. The method according to any one of the preceding items, wherein the unicellular organism is a pathogenic bacterium.
4. The method according to item 3, wherein the pathogenic bacterium is a gram positive or gram negative bacteria.
5. The method according to item 4, wherein the gram positive bacterium is of the phylum firmicutes.
6. The method according to item 5, wherein the firmicute is of a genus selected from the group consisting of Bacillus ssp., Staphylococcus ssp., Streptococcus ssp., Clostridium ssp. and Listeria ssp.
7. The method according to any one of the preceding items, wherein the binding moiety comprises at least one near iron transporter (NEAT) domain.
8. The method according to item 7, wherein the at least one NEAT domain comprises a sequence selected from the group consisting of:
   i. SEQ ID NO:1, SEQ ID NO:2, SEQ ID NO:3, SEQ ID NO:4, SEQ ID NO:5, SEQ ID NO:6, and combinations thereof, or
   ii. a NEAT domain having at least 75% sequence identity to the full-length sequences of any one of SEQ ID NOs:1-6 and combinations thereof.
9. The method according to item 8, wherein the NEAT domain of ii) has at least 90% sequence identity to the full-length sequences of any one of SEQ ID NOs:1-6 and combinations thereof.
10. The method according to any one of items 7-8, wherein the NEAT domain comprises a sequence selected from the group consisting of:
   i. SEQ ID NO:1, SEQ ID NO:2, SEQ ID NO:3, and combinations thereof, or
   ii. a NEAT domain having at least 75% sequence identity to the full-length sequence of any one of SEQ ID NOs:1-3 and combinations thereof.
11. The method according to item 10, wherein the NEAT domain of ii) has at least 90% sequence identity to the full-length sequence of any one of SEQ ID NOs:1-3 and combinations thereof.
12. The method according to any one of items 7-11, wherein the NEAT domain comprises:
   i. SEQ ID NO:2, or
   ii. a NEAT domain having at least 90% sequence identity to the full-length sequence of SEQ ID NO:2.
13. The method according to any one of items 7-12, wherein the NEAT domain comprises:
   i. SEQ ID NO:1 or a NEAT domain having at least 90% sequence identity to the full-length sequence SEQ ID NO:1, and
   ii. SEQ ID NO:2 or a NEAT domain having at least 90% sequence identity to the full-length sequence SEQ ID NO:2.
14. The method according to any one of items 7-13, wherein the NEAT domain consists of:
   i. SEQ ID NO:1 or a NEAT domain having at least 90% sequence identity to the full-length sequence SEQ ID NO:1, and
   ii. SEQ ID NO:2, or a NEAT domain having at least 90% sequence identity to the full-length sequence SEQ ID NO:2.
15. The method according to any one of items 7-12, wherein the NEAT domain is selected from the group consisting of:
   i. SEQ ID NO:1, SEQ ID NO:2, SEQ ID NO:3 and combinations thereof, or
   ii. a NEAT domain having at least 90% sequence identity to the full-length sequence of any one of SEQ ID NOs:1-3 and combinations thereof.
16. The method according to item 7, wherein the NEAT domain comprises:
   i. SEQ ID NO:7 or a NEAT domain having at least 90% sequence identity to the full-length sequence of SEQ ID NO:7, or
   ii. SEQ ID NO:8 or a NEAT domain having at least 90% sequence identity to the full-length sequence of SEQ ID NO:8.
17. The method according to item 16, wherein the NEAT domain is selected from the group consisting of:
   i. SEQ ID NO:7 or a NEAT domain having at least 90% sequence identity to the full-length sequence of SEQ ID NO:7, or
   ii. SEQ ID NO:8 or a NEAT domain having at least 90% sequence identity to the full-length sequence of SEQ ID NO:8.
18. The method according to any one of the preceding items, wherein the at least one non-mammalian protein or protein fragment comprises a coupling moiety.
19. The method according to item 18, wherein the coupling moiety is selected from a cysteine residue, an unnatural amino acid, an amino acid linker, an amino group, a consensus sequence and polyethylene glycol (PEG).
20. The method according to any one of items 18-19, wherein the coupling moiety is recombinantly introduced into the at least one non-mammalian protein or protein fragment.
21. The method according to any one of items 18-20, wherein the coupling moiety is a cysteine residue recombinantly introduced into the at least one non-mammalian protein or protein fragment.
22. The method according to any one of items 18-21, wherein the at least one non-mammalian protein or protein fragment is immobilized on the solid support via the coupling moiety.
23. The method according to any one of the preceding items, wherein the at least one non-mammalian protein or protein fragment further comprises a His-tag.
24. The method according to any one of the preceding items, wherein the sample is selected from the group consisting of whole blood, serum, plasma, and red cell lysates.
25. The method according to any one of the preceding items, wherein the solid support is of a material selected from the group consisting of plastic, glass, metal, silica, polymers, Sepharose, dextran, carboxymethyl dextran, and combinations thereof.
26. The method according to any one of the preceding items, wherein the solid support is in a form selected from the group consisting of a test tube, a resin, a microtiter plate, and a bead.
27. The method according to item 26, wherein the solid support is a test tube.
28. The method according to item 27, wherein the test tube is made of plastic.
29. The method according to item 28, wherein the test tube is a blood collection tube made of plastic.
30. The method according to any one of the preceding items, wherein the separation is accomplished by a technique selected from the group consisting of washing, eluting, filtration, centrifugation, magnetic separation, and combinations thereof.
31. The method according to any one of the preceding items, wherein the concentration of hemoglobin in the sample after step c) according to item 1 is reduced to at most 200 mg/dl, such as at most 150 mg/dl, such as at most 125 mg/dl, such as at most 100 mg/dl, such as at most 75 mg/dl, such as at most 50 mg/dl, such as at most 25 mg/dl.
32. The method according to item 31, wherein the concentration of hemoglobin in the sample is reduced to at most 100 mg/dl.
33. The method according to item 32, wherein the concentration of hemoglobin in the sample is reduced to at most 25 mg/dl.
34. A solid support to which is immobilized at least one non-mammalian protein or protein fragment derived from a unicellular organism and comprising a binding moiety which specifically binds hemoglobin and/or the complex of hemoglobin with haptoglobin.
35. The solid support according to item 34, wherein the unicellular organism is selected from the group consisting of bacteria, fungi and protozoa.
36. The solid support according to any one of items 34-35, wherein the unicellular organism is a pathogenic bacteria.
37. The solid support according to item 36, wherein the pathogenic bacteria is a gram positive or gram negative bacteria.
38. The solid support according to item 37, wherein the gram positive bacteria is of the phylum firmicutes.
39. The solid support according to item 38, wherein the firmicute is of a genus selected from the group consisting of Bacillus ssp., Staphylococcus ssp., Streptococcus ssp., Clostridium ssp. and Listeria ssp.
40. The solid support according to any one of items 34-39, wherein the binding moiety comprises at least one near iron transporter (NEAT) domain.
41. The solid support according to item 40, wherein the at least one NEAT domain comprises a sequence selected from the group consisting of:
   i. SEQ ID NO:1, SEQ ID NO:2, SEQ ID NO:3, SEQ ID NO:4, SEQ ID NO:5, SEQ ID NO:6, and combinations thereof, or
   ii. a NEAT domain having at least 75% sequence identity to the full-length sequences of any one of SEQ ID NOs:1-6 and combinations thereof.
42. The solid support according to item 41, wherein the NEAT domain of ii) has at least 90% sequence identity to the full-length sequences of any one of SEQ ID NOs:1-6 and combinations thereof.
43. The solid support according to any one of items 40-41, wherein the NEAT domain comprises a sequence selected from the group consisting of:
   i. SEQ ID NO:1, SEQ ID NO:2, SEQ ID NO:3, and combinations thereof, or
   ii. a NEAT domain having at least 75% sequence identity to the full-length sequence of any one of SEQ ID NOs:1-3 and combinations thereof.
44. The solid support according to item 43, wherein the NEAT domain of ii) has at least 90% sequence identity to the full-length sequence of any one of SEQ ID NOs:1-3 and combinations thereof.
45. The solid support according to any one of items 40-44, wherein the NEAT domain comprises:
   i. SEQ ID NO:2, or
   ii. a NEAT domain having at least 90% sequence identity to the full-length sequence of SEQ ID NO:2.
46. The solid support according to any one of items 40-45, wherein the NEAT domain comprises:
   i. SEQ ID NO:1 or a NEAT domain having at least 90% sequence identity to the full-length sequence SEQ ID NO:1, and
   ii. SEQ ID NO:2 or a NEAT domain having at least 90% sequence identity to the full-length sequence SEQ ID NO:2.
47. The solid support according to any one of items 40-46, wherein the NEAT domain consists of:
   i. SEQ ID NO:1 or a NEAT domain having at least 90% sequence identity to the full-length sequence SEQ ID NO:1, and
   ii. SEQ ID NO:2, or a NEAT domain having at least 90% sequence identity to the full-length sequence SEQ ID NO:2.
48. The solid support according to any one of items 40-45, wherein the NEAT domain is selected from the group consisting of:
   i. SEQ ID NO:1, SEQ ID NO:2, SEQ ID NO:3 and combinations thereof, or
   ii. a NEAT domain having at least 90% sequence identity to the full-length sequence of any one of SEQ ID NOs:1-3 and combinations thereof.
49. The solid support according to item 40, wherein the NEAT domain comprises:
   i. SEQ ID NO:7 or a NEAT domain having at least 90% sequence identity to the full-length sequence of SEQ ID NO:7, or
   ii. SEQ ID NO:8 or a NEAT domain having at least 90% sequence identity to the full-length sequence of SEQ ID NO:8.
50. The solid support according to item 49, wherein the NEAT domain is selected from the group consisting of:
   i. SEQ ID NO:7 or a NEAT domain having at least 90% sequence identity to the full-length sequence of SEQ ID NO:7, or
   ii. SEQ ID NO:8 or a NEAT domain having at least 90% sequence identity to the full-length sequence of SEQ ID NO:8.
51. The solid support according to any one of items 34-50, wherein the at least one non-mammalian protein or protein fragment comprises a coupling moiety.
52. The solid support according to item 51, wherein the coupling moiety is selected from a cysteine residue, an unnatural amino acid, an amino acid linker, an amino group, a consensus sequence and polyethylene glycol (PEG).
53. The solid support according to any one of items 51-52, wherein the coupling moiety is recombinantly introduced into the at least one non-mammalian protein or protein fragment.
54. The solid support according to any one of items 51-53, wherein the coupling moiety is a cysteine residue recombinantly introduced into the at least one non-mammalian protein or protein fragment.
55. The solid support according to any one of items 51-54, wherein the at least one non-mammalian protein or protein fragment is immobilized on the solid support via the coupling moiety.
56. The solid support according to any one of items 34-55, wherein the at least one non-mammalian protein or protein fragment further comprises a His-tag.
57. The solid support according to any one of items 34-56, wherein the solid support is of a material selected from the group consisting of plastic, glass, metal, silica, polymers, Sepharose, dextran, carboxymethyl dextran, and combinations thereof.
58. The solid support according to any one of items 34-57, wherein the solid support is in a form selected from the group consisting of a test tube, a resin, a microtiter plate, and a bead.
59. A container comprising a solid support according to any one of items 34-57.
60. The container according to item 59, wherein the surface of the container is coated with the solid support.
61. The container according to any one of items 59-60, wherein the container is a test tube.
62. The container according to any one of items 59-61, wherein the container is made of plastic.
63. The container according to any one of items 59-62, wherein the container is a blood collection tube made of plastic.
64. A kit for removal of hemoglobin from a sample, the kit comprising:
   i. a container according to any one of items 59-63, and
   ii. instructions for use.
65. Use of a non-mammalian protein or protein fragment for removal of hemoglobin from a sample, wherein said non-mammalian protein or protein fragment is derived from a unicellular organism and comprises a binding moiety which specifically binds hemoglobin and/or the complex of hemoglobin with haptoglobin.
66. The use according to item 65, wherein the unicellular organism is selected from the group consisting of bacteria, fungi and protozoa.
67. The use according to any one of items 65-66, wherein the unicellular organism is a pathogenic bacteria.
68. The use according to item 67, wherein the pathogenic bacteria is a gram positive or gram negative bacteria.
69. The use according to item 68, wherein the gram positive bacteria is of the phylum firmicutes.
70. The use according to item 69, wherein the firmicute is of a genus selected from the group consisting of Bacillus ssp., Staphylococcus ssp., Streptococcus ssp., Clostridium ssp. and Listeria ssp.
71. The use according to any one of items 65-70, wherein the binding moiety comprises at least one near iron transporter (NEAT) domain.
72. The use according to item 71, wherein the at least one NEAT domain comprises a sequence selected from the group consisting of:
   i. SEQ ID NO:1, SEQ ID NO:2, SEQ ID NO:3, SEQ ID NO:4, SEQ ID NO:5, SEQ ID NO:6, and combinations thereof, or
   ii. a NEAT domain having at least 75% sequence identity to the full-length sequences of any one of SEQ ID NOs:1-6 and combinations thereof.
73. The use according to item 72, wherein the NEAT domain of ii) has at least 90% sequence identity to the full-length sequences of any one of SEQ ID NOs:1-6 and combinations thereof.
74. The use according to any one of items 71-72, wherein the NEAT domain comprises a sequence selected from the group consisting of:
   i. SEQ ID NO:1, SEQ ID NO:2, SEQ ID NO:3, and combinations thereof, or
   ii. a NEAT domain having at least 75% sequence identity to the full-length sequence of any one of SEQ ID NOs:1-3 and combinations thereof.
75. The use according to item 74, wherein the NEAT domain of ii) has at least 90% sequence identity to the full-length sequence of any one of SEQ ID NOs:1-3 and combinations thereof.
76. The use according to any one of items 71-75, wherein the NEAT domain comprises:
   i. SEQ ID NO:2, or
   ii. a NEAT domain having at least 90% sequence identity to the full-length sequence of SEQ ID NO:2.
77. The use according to any one of items 71-76, wherein the NEAT domain comprises:
   i. SEQ ID NO:1 or a NEAT domain having at least 90% sequence identity to the full-length sequence SEQ ID NO:1, and
   ii. SEQ ID NO:2 or a NEAT domain having at least 90% sequence identity to the full-length sequence SEQ ID NO:2.
78. The use according to item 71-77, wherein the NEAT domain consists of:
   i. SEQ ID NO:1 or a NEAT domain having at least 90% sequence identity to the full-length sequence SEQ ID NO:1, and
   ii. SEQ ID NO:2, or a NEAT domain having at least 90% sequence identity to the full-length sequence SEQ ID NO:2.
79. The use according to any one of items 71-76, wherein the NEAT domain is selected from the group consisting of:
   i. SEQ ID NO:1, SEQ ID NO:2, SEQ ID NO:3 and combinations thereof, or
   ii. a NEAT domain having at least 90% sequence identity to the full-length sequence of any one of SEQ ID NOs:1-3 and combinations thereof.
80. The use according to item 71, wherein the NEAT domain comprises:
   i. SEQ ID NO:7 or a NEAT domain having at least 90% sequence identity to the full-length sequence of SEQ ID NO:7, or
   ii. SEQ ID NO:8 or a NEAT domain having at least 90% sequence identity to the full-length sequence of SEQ ID NO:8.
81. The use according to item 80, wherein the NEAT domain is selected from the group consisting of:
   i. SEQ ID NO:7 or a NEAT domain having at least 90% sequence identity to the full-length sequence of SEQ ID NO:7, or
   ii. SEQ ID NO:8 or a NEAT domain having at least 90% sequence identity to the full-length sequence of SEQ ID NO:8.
82. The use according to any one of items 65-81, wherein the at least one non-mammalian protein or protein fragment comprises a coupling moiety.
83. The use according to item 82, wherein the coupling moiety is selected from a cysteine residue, an unnatural amino acid, an amino acid linker, an amino group, a consensus sequence and polyethylene glycol (PEG).
84. The use according to any one of items 82-83, wherein the coupling moiety is recombinantly introduced into the at least one non-mammalian protein or protein fragment.
85. The use according to any one of items 82-84, wherein the coupling moiety is a cysteine residue recombinantly introduced into the at least one non-mammalian protein or protein fragment.
86. The use according to any one of items 82-85, wherein the at least one non-mammalian protein or protein fragment is immobilized on the solid support via the coupling moiety.
87. The use according to any one of items 65-86, wherein the at least one non-mammalian protein or protein fragment further comprises a His-tag.
88. The use according to any one of items 65-87, wherein the sample is selected from the group consisting of whole blood, serum, plasma, and red cell lysates.
89. The use according to any one of items 65-88, wherein the concentration of hemoglobin in the sample after use is reduced to at most 200 mg/dl, such as at most 150 mg/dl, such as at most 125 mg/dl, such as at most 100 mg/dl, such as at most 75 mg/dl, such as at most 50 mg/dl, such as at most 25 mg/dl.
90. The use according to item 89, wherein the concentration of hemoglobin in the sample is reduced to at most 100 mg/dl.
91. The use according to item 90, wherein the concentration of hemoglobin in the sample is reduced to at most 25 mg/dl.

The invention will now be described in further details in the following non-limiting examples.

### EXAMPLES

### Example 1: Generation of IsdH-N-Sepharose Through Coupling to Primary Amines

The DNA sequence encoding IsdH^{N1} (residues 86-229) with an added N-terminal His6 tag and a thrombin cleavage site was cloned into the Ndel and BamHI sites (Genscript) of the pET-22b(+) (Novagen) vector. The sequence encoding IsdH^{N2N3} (residues 321-655) was cloned in the XhoI and BamHI sites of pET-15b (Novagen) in line with the vector encoded His6 tag and purified over nickel affinity, anion exchange chromatography. IsdH^{N1N2N3} (residues 82-655) was expressed and purified according to the same method as used for IsdH^{N2N3} purification. The protein purity was assessed by SDS gel electrophoresis.

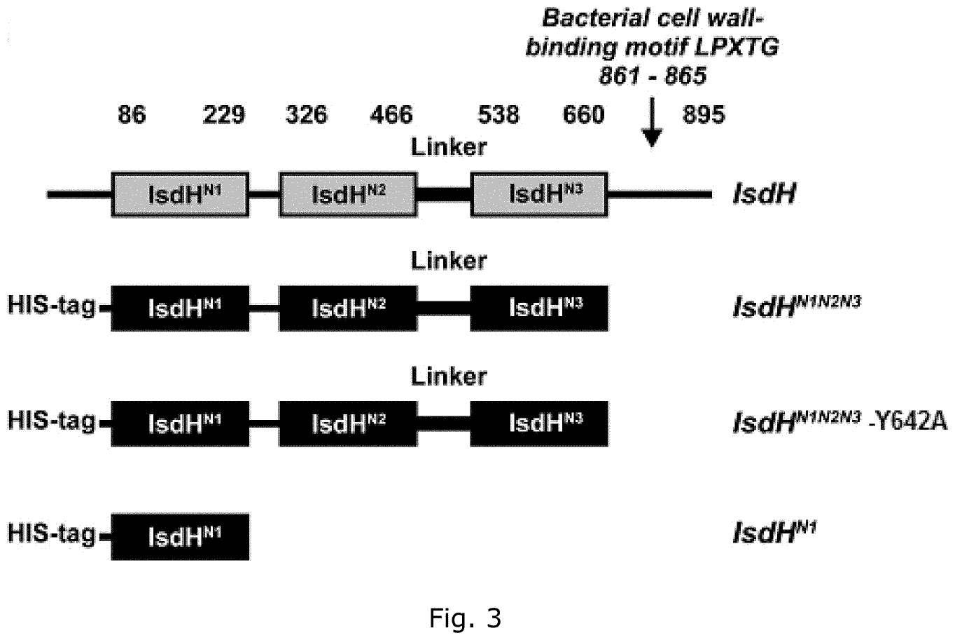

An overview of the different IsdH constructs used (IsdH N1N2N3, IsdH N1 and IsdH N1N2N3 Y642A) can be found in FIG. 3.

To couple the IsdH derivatives to Sepharose, 2 ml 5 mg/ml IsdH proteins dissolved in 0.1 M NaHCO₃ pH 8.3 containing 0.5 M NaCl were incubated with 2 ml of CNBr-activated Sepharose washed according to the manufacturer's instructions (GE Lifesciences, Brøndby, Denmark). In addition sham coupled Sepharose was made. The mixture was incubated for 3 hours at room temperature. The absorbance at 280 nm was measured at the start and the end of incubation. Remaining amine-reactive groups were quenched by adding 1 M Tris-HCl, pH 8.0 to a final concentration of 100 mM. The column materials were washed thoroughly with 10 mM Hepes, 150 mM NaCl pH 7.5 and stored at 4° C.

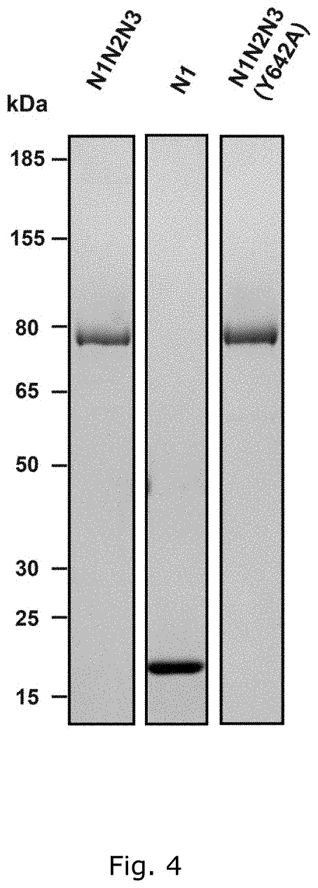

The three constructs were successfully expressed and purified. An SDS-PAGE showing the final purified proteins can be seen in FIG. 4. The absorbance dropped from the start of the conjugation of proteins to CNBr-Sepharose to the end, by more than 90%, indicating an effective conjugation.

Conclusion:

IsdH was efficiently conjugated to Sepharose through reaction with primary amines of IsdH.

### Example 2: Removal of Hemoglobin from Plasma Using Amine-Conjugated IsdH-N-Sepharose

Human plasma was freshly obtained from a healthy donor, collected in EDTA coated vacuum tubes. Hemoglobin (Sigma-Aldrich) was added to plasma to a final concentration of 3 g/L.

To 2 ml of either raw plasma or plasma spiked with hemoglobin was added an equal volume of the coupled Sepharoses described in example 1. The samples were mixed and incubated for 15 minutes on ice, centrifuged at 4000×g and supernatants removed for analysis.

For all the samples the following biochemical plasma parameters; H-index, alanine transaminase (ALAT), aspartate transaminase (ASAT), and albumin were determined using a Roche Diagnostics Cobas 6000 instrument according to the manufacturer's instructions. Briefly, for preparation of the baseline pools, heparinized plasma or serum were selected from samples with the lowest indexes of hemolysis, icterus and lipemia, measured on Modular® analysers and selected using the MPL® middleware (Roche Diagnostics). For the two ranges of overloaded pools, the H-index were measured in duplicate on two Modular P800®, by bichromatic spectrophotometry at 570 nm and 600 nm wavelength pair. The H-index were measured using the following formula: H-index=1/scaling factor for hemoglobin*(A570−600 nm−correcting factor for hemoglobin measurement for lipemia*A660−700 nm), in order to compensate the spectral overlap due to lipemia.

Measurement of ASAT is one of the most hemolysis sensitive assays performed in the routine laboratory, whereas albumin is insensitive to hemolysis and functions as an internal standard. In addition, ALAT was used as a secondary internal standard, since low levels of hemoglobin (e.g. ˜H-index<200) is reported not to affect the measurement of ALAT.

The plasma parameters measured before and after addition of hemoglobin and/or exposure to the different Sepharoses can be found in table 1:

TABLE 1
Raw data of plasma parameters determined, average of triplicate.

| | Added | | | | |
|---|---|---|---|---|---|
| | hemoglobin | Albumin | ALAT | ASAT | H-index |
| Sepharose type | g/l | g/l | U/l | U/l | mg/dl |
| No Sepharose | 0 | 43.0 | 21.0 | 33.3 | 7 |
| | 3 | 39.2 | 19.9 | n.a. | 80 |
| Sham Seph | 0 | 14.8 | 7.8 | 11.2 | 0 |
| | 3 | 15.5 | 8.1 | n.a. | 25 |
| N1 Seph | 0 | 19.0 | 9.3 | 13.7 | 0 |
| | 3 | 19.1 | 9.7 | n.a. | 29 |
| N1N2N3- | 0 | 16.1 | 8.6 | 11.7 | 0 |
| Y642A-Seph | 3 | 15.6 | 8.1 | 23.1 | 8 |
| N1N2N3-Seph | 0 | 20.5 | 9.9 | 14.8 | 0 |
| | 3 | 17.7 | 9.2 | 25.9 | 12 |

N.a. means that the value cannot be determined due to a too high H-index.

It is shown that the full-length IsdH conjugated Sepharoses more efficiently removes hemoglobin than sham or N1 conjugated Sepharoses, as reflected in a larger drop in the H-index. Further the Y642A mutant is more efficient than the wild type.

Of the determined parameters ASAT was sensitive to hemoglobin, whereas albumin and ALAT were not. Since addition of Sepharose leads to dilution of the plasma sample, a dilution factor for each sample was determined by correction of albumin back to the original level of the No Sepharose control. Thereby the following levels (not including H-index) of plasma parameters compensated for dilution were obtained:

TABLE 2
Data of plasma parameters determined compensated for dilution using
albumin as reference, average of triplicate.

| | Added | | | |
|---|---|---|---|---|
| | hemoglobin | Albumin | ALAT | ASAT |
| Sepharose type | g/l | g/l | U/l | U/l |
| No Sepharose | 0 | 43 | 21 | 33.3 |
| | 3 | 43.0 | 21.8 | n.a. |
| Sham Seph | 0 | 43.0 | 22.7 | 32.5 |
| | 3 | 43.0 | 22.5 | n.a. |
| N1 Seph | 0 | 43.0 | 21.0 | 31.0 |
| | 3 | 43.0 | 21.8 | n.a. |
| N1N2N3-Y642A-Seph | 0 | 43.0 | 23.0 | 31.2 |
| | 3 | 43.0 | 22.3 | 63.7 |
| N1N2N3-Seph | 0 | 43.0 | 20.8 | 31.0 |
| | 3 | 43.0 | 22.4 | 62.9 |

N.a. means that the value cannot be determined due to a too high H-index.

Conclusion:

As can be seen from Tables 1 and 2, exposure of hemolysed plasma to Sepharose conjugated with either IsdH N1N2N3 wt or Y642A mutant, significantly lowered the H-index and made a determination of ASAT possible. Further, normalization using albumin as reference restored ALAT values to that of the original, indicating that neither albumin nor ALAT determination were affected by exposure to the Sepharoses, and that normalization using albumin as reference is warranted. It was shown that non-specific interactions with the Sepharose leading to changes in parameters is not a problem.

### Example 3: Expression of IsdH Variants for Site-Specific Conjugation of IsdH to Thiopropyl-Sepharose and Test of Hb Binding Efficiency Compared to HemogloBind

In order to retain high reactivity of IsdH after conjugation to a solid-phase matrix, we introduce a reactive sulfhydryl group in the proteins by insertion of a cysteine residue, which IsdH does not have in its original sequence. This allow specific orientation of IsdH after conjugation in relation to the matrix, and further ensures that the amino acid residues involved in binding of hemoglobin or the complex of hemoglobin with haptoglobin is not altered by immobilization.

Furthermore, since hemoglobin binding affinity resides in IsdH domains 1 and 2 a construct including only domains 1 and 2 is expressed and tested.

Plasmids with either a C- or N-terminal Cys residue are constructed, expressed in E. co/i, and purified. Purity and affinity towards hemoglobin or the complex of hemoglobin with haptoglobin is determined using surface plasmon resonance.

Taking advantage of the introduced sulfhydryl we conjugate IsdH to thiopropyl-Sepharose and show it to be active in binding hemoglobin and the complex of hemoglobin with haptoglobin, including estimating maximum binding capacity. The generated IsdH-thiopropyl-Sepharose matrix is termed IsdH-C-Seph.

Synthesis of IsdH-Thiopropyl-Sepharose (IsdH-C-Seph)

An amino-terminal cysteine residue was introduced in IsdH Tyr642Ala using the Q5 Site-Directed Mutagenesis kit (New England Biolabs, Ipswich, Mass., USA) and expressed and purified as wild type IsdH, but added 10 mm dithiothreitol in the buffer for ion-exchange.

IsdH N1N2N3-Y642A-Cys (SEQ ID NO: 23) was coupled to thiopropyl-Sepharose as follows:

IsdH N1N2N3-Y642A-Cys in 150 mM NaCl, 10 mM Hepes pH 7.4 was added dithiothreitol to 50 mM, incubated at room temperature for 15 minutes and gelfiltrated into 500 mM NaCl, 1 mM EDTA, 100 mM Tris-HCl pH 7.5. 20 mg of IsdH N1N2N3-Y642A-Cys was added per ml of thiopropyl-Sepharose and incubated for 5 hours. Added 1 mM cysteine and incubated for 1 hour at room temperature. The column material was then washed with PBS, making a 1:1 slurry of Sepharose and PBS.

Test of Hb Binding Capacity of IsdH-C-Seph

Erythrocytes isolated from fresh blood were lysed consecutive freeze-thaw cycles as follows. The erythrocyte containing cell layer of a blood sample was isolated, added a double volume of isotonic salt water and subjected to four freeze-thaw cycles in dry ice-water bath. The hemolysate was centrifuged at 2000×g for 7 minutes, and the supernatant isolated. The hemoglobin concentration of the hemolysate supernatant was determined using a using Roche Cobas 8000/c701 and Cobas 8000/e802 analyzers (Roche Diagnostics GmbH, Mannheim, Germany) with dedicated Cobas 8000 reagents.

Plasma free of hemolysis was adjusted to specified hemoglobin concentrations by adding the supernatant containing the hemolysate. Plasma with increasing hemoglobin concentrations was mixed with an equal volume of Sepharose. Samples were incubated for 30 minutes at room temperature and centrifuged at 1.000×g for 5 minutes. Supernatant was removed and hemoglobin concentration determined. The commercial product for hemoglobin removal from plasma HemogloBind (Biotech Support Group LLC, Monmouth Junction, New Jersey, USA) was also tested. The procedure was carried out according to the manufacturer's instruction.

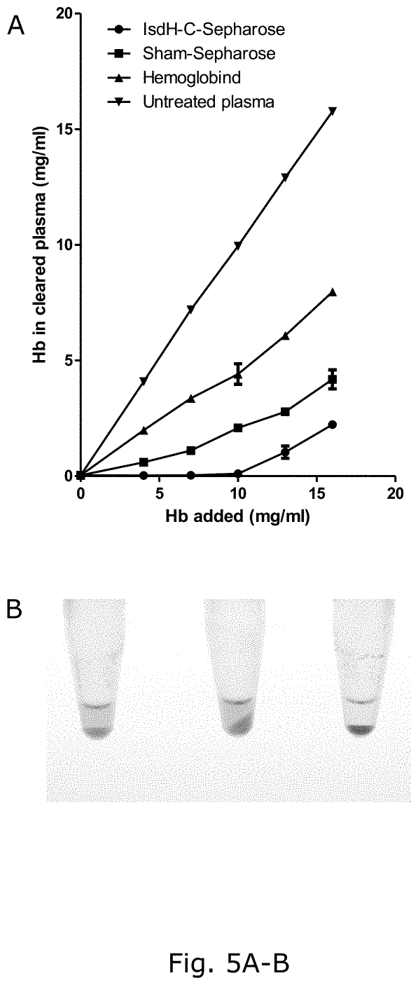

The efficiency of IsdH-C-Sepharose, Sham-Sepharose and HemogloBind in hemoglobin removal compared to an untreated plasma sample are depicted in FIG. 5A, values are of triplicate samples with standard deviation. As can be seen, IsdH-C-Sepharose is significantly better than the other constructs, and can remove all hemoglobin from the plasma samples at concentrations up to 10 mg/ml.

FIG. 5B shows the three column materials after incubation with 3 mg/ml hemoglobin plasma and centrifugation. From left to right the vials contain: Sham-Sepharose, HemogloBind, IsdH-C-Sepharose. It can clearly be seen that only IsdH-C-Sepharose concentrates the hemoglobin in the column material phase.

Conclusion:

The example shows that IsdH can be expressed as a single cysteine mutant that through the reactive sulfhydryl group of the cysteine can be conjugated to a solid support (here Sepharose). The IsdH-C-Seph matrix efficiently removes hemoglobin from a sample.

### Example 4: Comparison of IsdH-C-Seph to HemoVoid and HemogloBind

Plasma samples are added hemoglobin to a certain H-index and purified using IsdH-C-Seph. A variety of plasma biochemical parameters are assayed on a routine hospital clinical biochemical unit, with the parameters being normalized to those of un-spiked plasma (i.e. without added hemoglobin).

In parallel the hemoglobin spiked plasma is cleaned using the two commercially available hemoglobin removal products Hemovoid™ and HemogloBind™ and the same plasma biochemical parameters is determined.

Preparation of Samples

Human plasma was freshly obtained from EDTA coated vacuum tubes. Hemolysate was added to a hemoglobin concentration of 3 g/L.

To 500 μl of either raw plasma or plasma added hemoglobin was added an 170 μl of either IsdH-C-Y642A-Sepharose or Sham-Sepharose. The samples were mixed and incubated for 30 minutes at room temperature, centrifuged at 1000×g for 5 minutes and supernatants removed for analysis. The commercial product for hemoglobin removal from plasma HemogloBind (Biotech Support Group LLC, Monmouth Junction, N.J., USA) was also tested on these samples. The procedure was carried out according to the manufacturer's instruction

For all the samples the blood biochemical parameters measurements were performed using Roche Cobas 8000/c701 and Cobas 8000/e802 analyzers (Roche Diagnostics GmbH, Mannheim, Germany) with dedicated Cobas 8000 reagents.

Test of Blood Biochemistry Parameters after Clearance of Hemoglobin

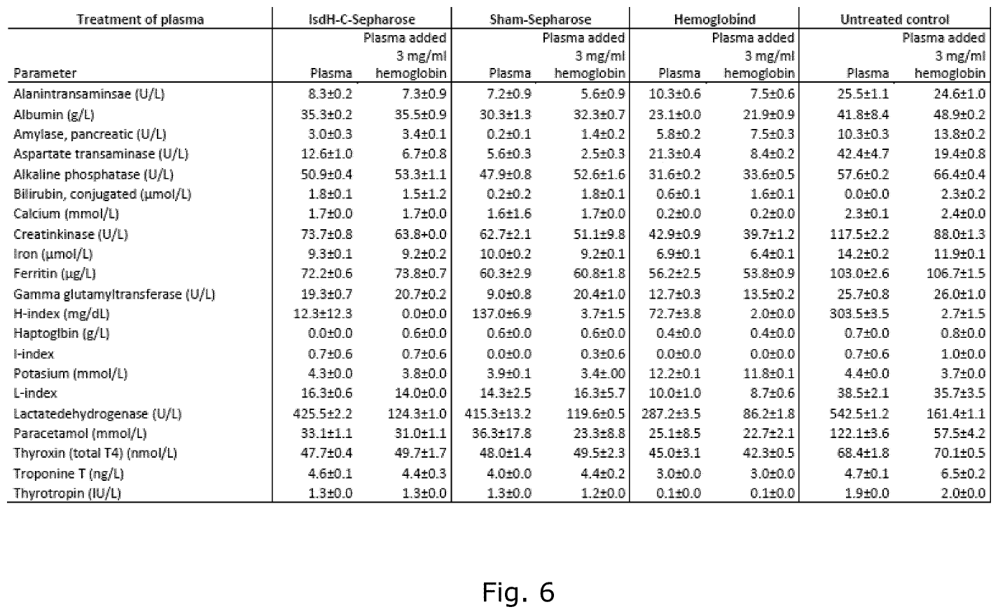

The plasma parameters measured before and after addition of Hb and/or exposure to the different Sepharoses can be found in FIG. 6 (table with data), the data are from triplicate removals/procedures.

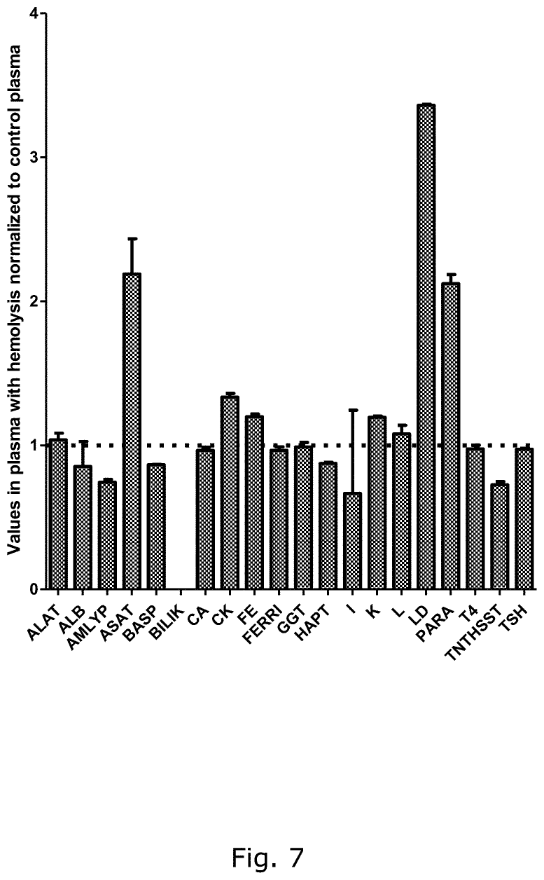

To illustrate which parameters are affected by hemolysis, a plot of the values determined in plasma with hemolysis normalized to same plasma without added hemolysate can be found in FIG. 7. It is apparent from the FIG. 7 that several parameters are affected by hemolysis.

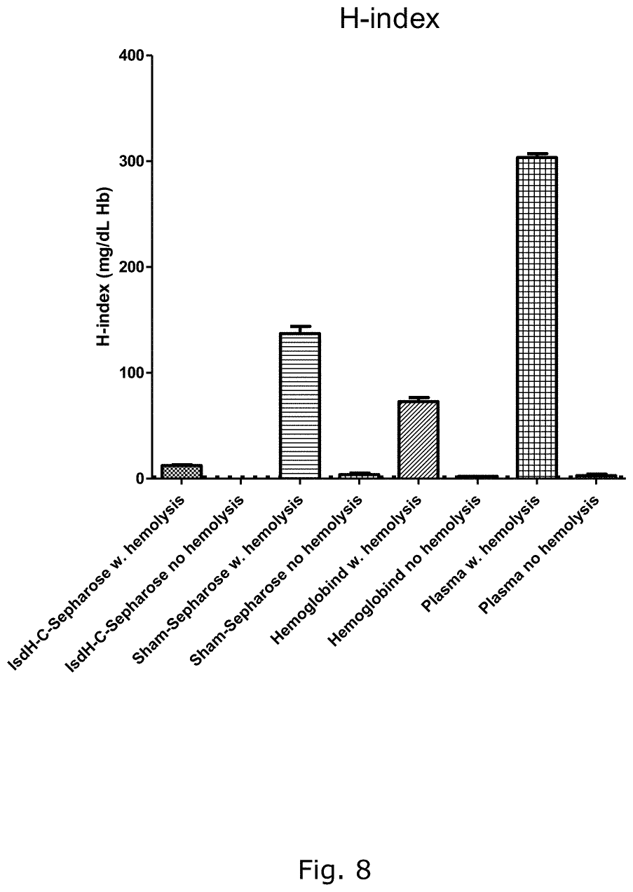

The effect on H-index, measured as hemoglobin in mg/dl, can be seen in FIG. 8. To compare the different plasma parameters, the albumin concentration was used for normalization of all samples, with plasma without hemolysate as reference. The H-index is efficiently reduced by IsdH-C-Sepharose.

In samples with high hemoglobin a substantial amount of haptoglobin will be in complex with hemoglobin. Efficient removal of hemoglobin from plasma or serum will thus demand that also hemoglobin bound to haptoglobin can be removed, which will be indicated by a concomitant reduction in both hemoglobin and haptoglobin.

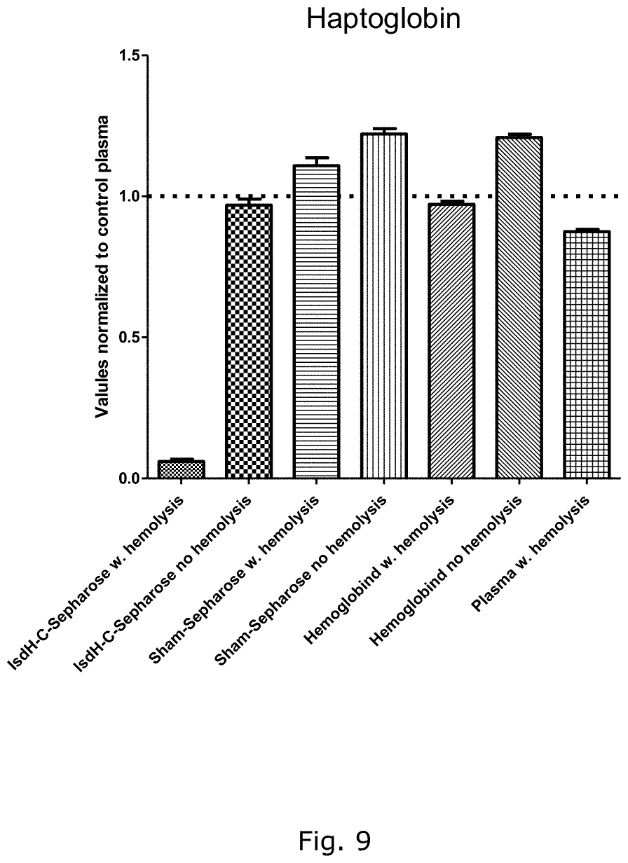

Notably, IsdH-C-Sepharose did not only reduce hemoglobin, but also substantially reduced haptoglobin, as can be seen in FIG. 9. In contrast, HemogloBind did not reduce Hp. The shortcomings of HemogloBind is most likely related to a shielding of hemoglobin, when in complex with haptoglobin, from binding to the material.

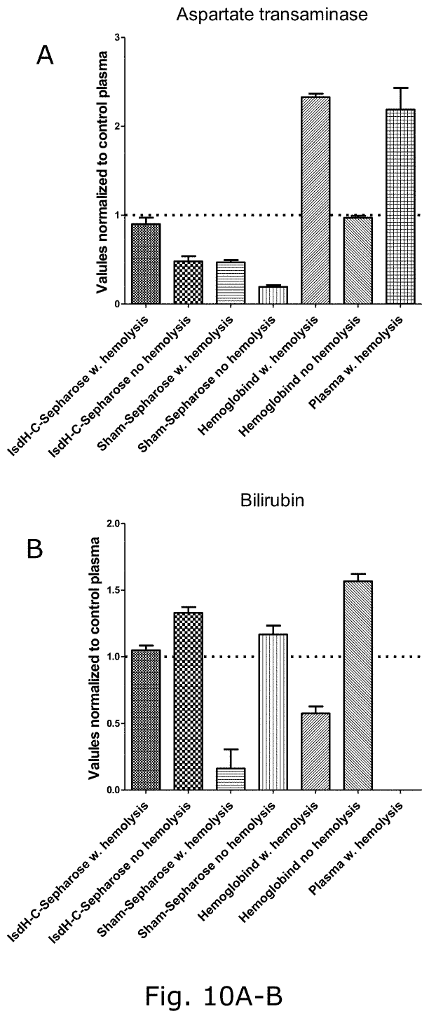

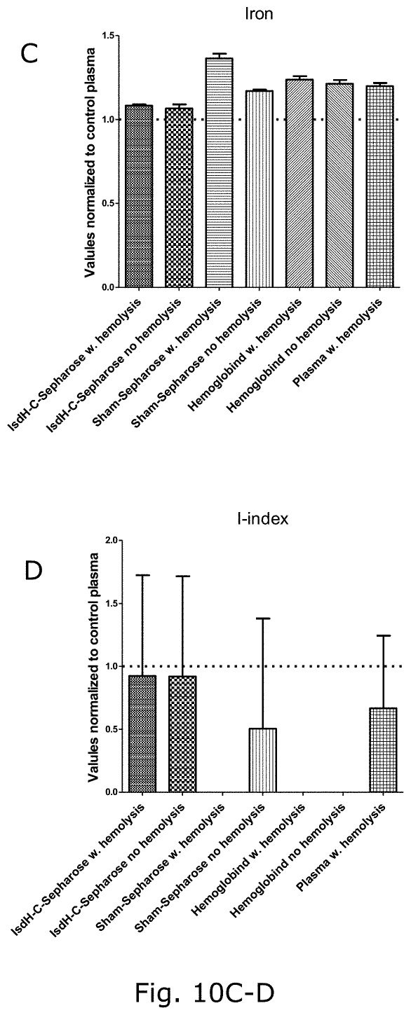

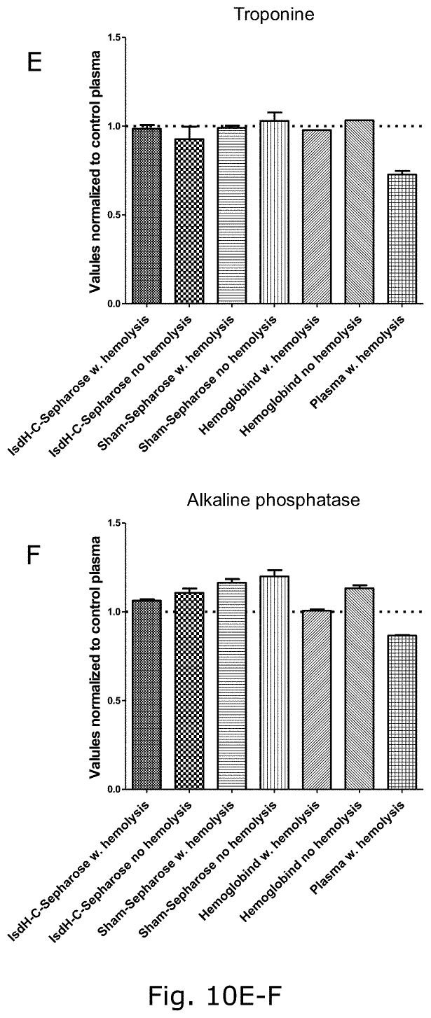

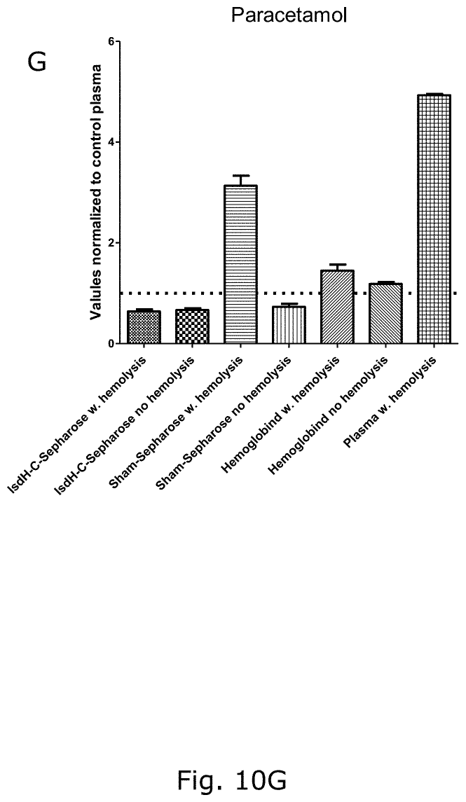

A range of blood biochemical parameters was affected by the efficiency with which hemoglobin was removed from the samples. Thus, IsdH-C-Sepharose mediated removal of hemoglobin enables correct measurements of aspartate transaminase, bilirubin, iron, I-index, troponine, alkaline phosphatase and paracetamol (see FIG. 10 A-G). Measurement are corrected for dilution of the samples. HemogloBind was considerably poorer in restoring values of e.g. aspartate transaminase, bilirubin, iron and I-index.

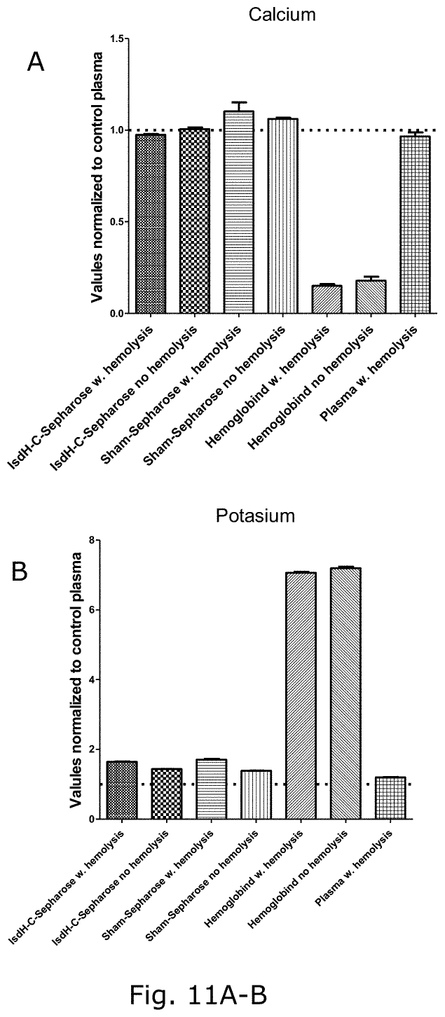

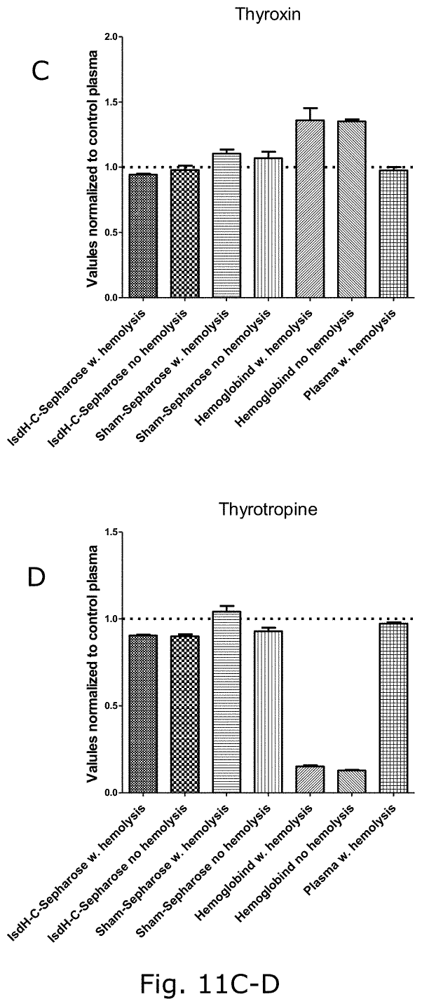

Further, a range of parameters was corrupted by HemogloBind, irrespective of hemoglobin was present or not. Those were most notably calcium and thyrotropine (see FIG. 11A-B), were unspecific binding to the material seems to be a problem and potassium and thyroxin (see FIG. 11C-D), were a substance interfering with measurements seems to be released from the material.

Conclusion:

The examples shows that IsdH-C-Sepharose efficiently reduced both hemoglobin and haptoglobin in the samples. In contrast, HemogloBind did not reduce hemoglobin as efficiently and failed to reduce haptoglobin from the samples.

Efficient removal of hemoglobin and haptoglobin enabled a range of blood biochemical parameters to be measured correctly using the IsdH-C-Sepharose construct for sample purification.

The example also shows that a range of parameters are unaffected by the IsdH-C-Seph matrix, whereas the HemogloBind™ matrix is subject to non-specific binding.

Furthermore, the IsdH-C-Seph matrix can handle higher H-index values than Hemovoid™ and HemogloBind™

### Example 5: Use of IsdH-C-Seph for Removal of Hemoglobin from Hemolysed Plasma Samples from Human Patients

Plasma samples from patients with severe hemolysis are obtained and hemoglobin is removed using the IsdH-C-Seph matrix. The H-index is measured before and after purification of the hemolysed samples and biochemical plasma parameters are determined.

Measurements of Random Plasma Samples with Hemolysis

To test how well hemoglobin could be removed from random patient samples, 9 anonymized samples with a hemolytic index too high for routine measurements of a range of parameters, were tested for hemoglobin removal and subsequently selected blood biochemistry parameters was determined.

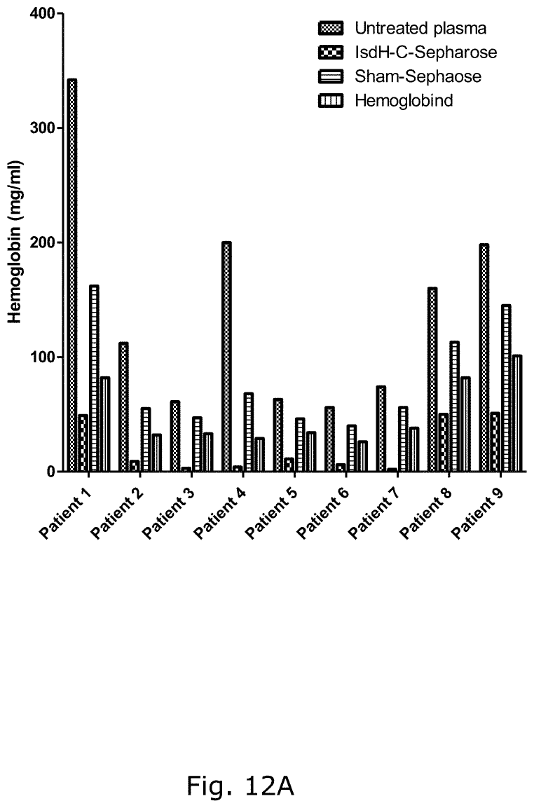

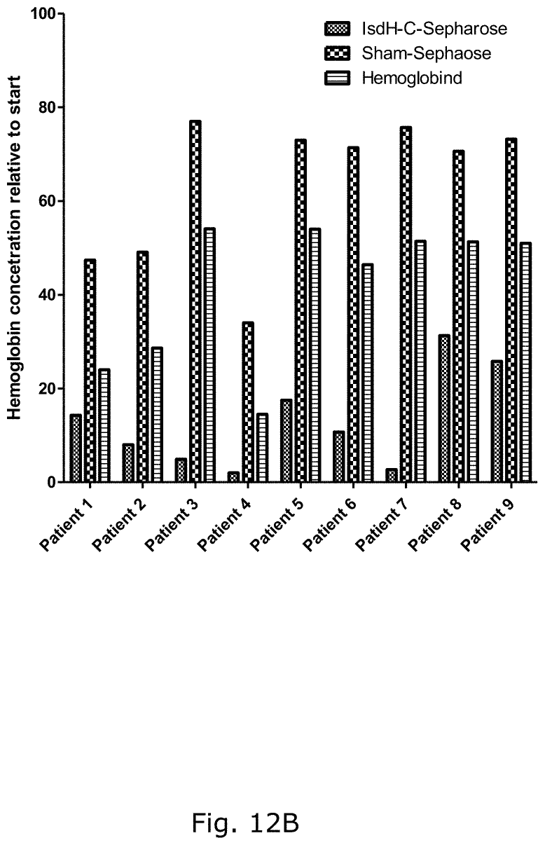

First 1 ml of each plasma sample with hemolysis was cleared for hemolysis using either 170 μl IsdH-C-Sepharose, 170 μl Sham-Sepharose or 500 μl HemogloBind slurry (see FIG. 12A). For easy comparison, the data from the different patients are also presented as percentage of initial hemoglobin remaining in the samples (see FIG. 12B).

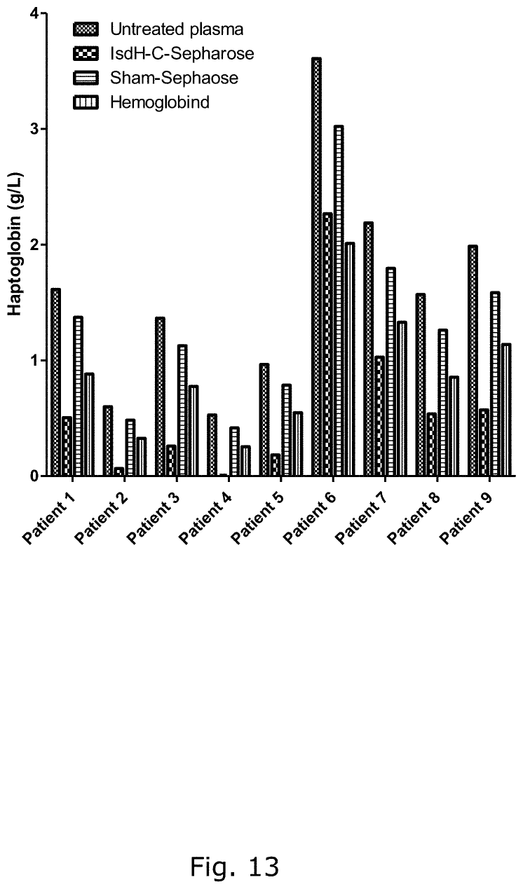

Measurements of the haptoglobin levels in the samples revealed that also haptoglobin levels were efficiently reduced using the IsdH-C-Sepharose construct (see FIG. 13). Thus, IsdH-C-Sepharose not only removes hemoglobin but also most efficiently removes haptoglobin, again reflecting that IsdH binds both hemoglobin and the complex of hemoglobin and haptoglobin.

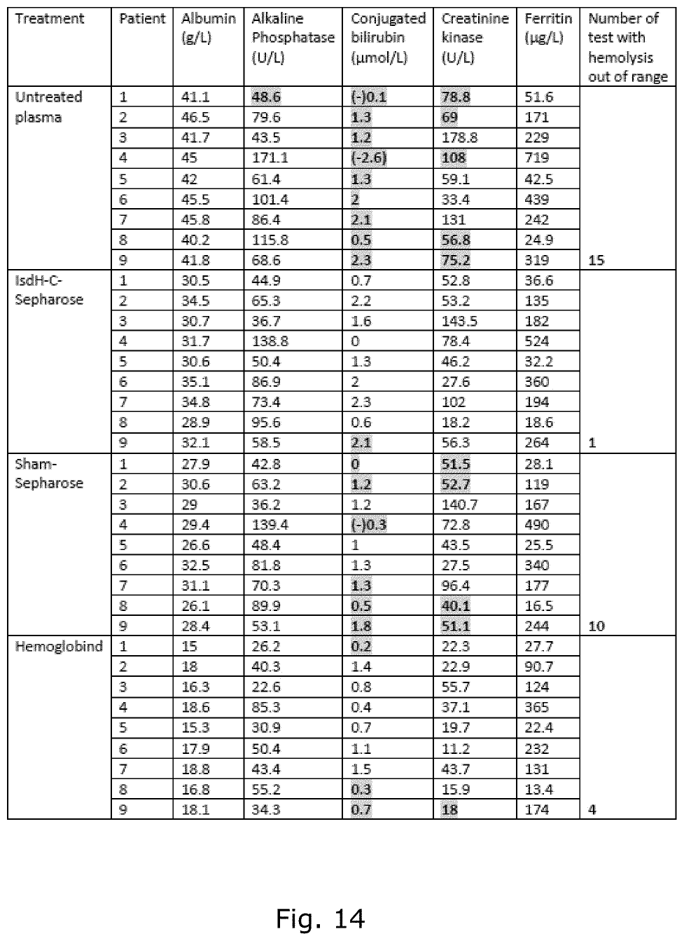

Furthermore, the following blood biochemical parameters was determined: albumin, alkaline phosphatase, conjugated bilirubin, creatinine kinase, and ferritin (see FIG. 14). Determinations that were automatically assigned by the Cobas system to be unfit for analysis due to too high hemolysis in the sample is in bold on grey background.

It is evident that IsdH-C-Sepharose enables the measurement of most samples. With the hemolysis range in the plasma samples selected, especially measurement of conjugated bilirubin and creatinine kinase is enabled. The measured albumin concentrations can be used for normalization.

Conclusion:

The example shows that the use of the IsdH-C-Seph matrix (and other non-mammalian proteins with affinity for hemoglobin and/or the complex of hemoglobin with haptoglobin) for removal of hemoglobin from hemolysed samples is applicable in a clinical setting.

The IsdH-C-Sepharose construct removed most efficiently hemoglobin and haptoglobin from the patient samples and consistently enabled measurement of a range blood biochemical parameters.

### Example 6: Use of Other Hemoglobin Binding Proteins for Removal of Hemoglobin from Hemolysed Samples

In addition to IsdH, the following proteins are cloned and expressed:
HtaA from Corynebacterium diphtheriae which contains a CR domain that binds hemoglobin and/or the complex of hemoglobin with haptoglobin
Shr from Streptococcus pyogenes which contains two NEAT domains
IsdB from Staphylococcus aureus which contains two NEAT domains

The expressed and purified proteins are conjugated to Sepharose and hemoglobin removal from plasma using the functionalized matrices is performed.

Expression of Proteins

The proteins IsdB, Htaa and Shr was expressed and captured on Ni-NTA affinity matrix as for IsdH. IsdB and Htaa were further purified using a Q-Sepharose (GE Healthcare, Brøndbyvester, Denmark) ion-exchange at pH 8 with a gradient from 25-500 mM NaCl. Shr was further purified on a SP-Sepharose (GE Healthcare, Brøndbyvester, Denmark) in 4 M urea, 25 mM Tris-HCl with a gradient from 10-1000 mM NaCl.

IsdH Y642A was prepared as described previously herein.

Affinity purified polyclonal anti-Hb IgY was made in chickens by the company Sanovo Biotech (Odense, Denmark) by immunizing with human hemoglobin and purifying anti-Hb IgY from eggs on a Hb Sepharose column, made as follows: 20 mg human Hb HO (Sigma-Aldrich, Brøndbyvester, Danmark) was dissolved in 50 mM NaHCO₃, 500 mM NaCl, pH 8.3 and mixed with 3.9 gram of CNBr-activated Sepharose prepared according to manufacturer's instructions (GE Healthcare, Brøndbyvester, Denmark) and incubated for 3 hours at room temperature, added Tris-HCl pH 8.0 to 100 mM, incubated overnight. Finally, the column material was washed with PBS pH 7.4.

Conjugation to CNBr-Activated Sepharose

10 mg of each protein in 100 mM NaHCO₃ pH 8.3 was added to 1 ml CNBr-activated Sepharose washed with 100 mM NaHCO₃. The coupling was allowed to run for 16 hours at 4° C. Degree of conjugation was accessed by measuring OD(280) of the supernatant, for all protein >95% of the protein had reacted with the matrix. The material was quenched by adding Tris-HCl pH 8.0 to 100 mM and incubate for 1 hour. The materials were then washed into PBS pH 7.4, with a final 1:1 slurry of Sepharose and PBS prepared. Thus, IsdH was linked to CNBr-activated Sepharose through an amino group (IsdH-N).

Test of Hemoglobin Clearance

Erythrocytes isolated from fresh blood were lysed consecutive freeze-thaw cycles as follows. The erythrocyte containing cell layer of a blood sample was isolated, added a double volume of isotonic salt water and subjected to four freeze-thaw cycles in dry ice-water bath. The hemolysate was centrifuged at 2000 G for 7 minutes, and the supernatant isolated. The hemoglobin concentration of the hemolysate supernatant was determined using a Roche Cobas 8000/c701 and Cobas 8000/e802 analyzers (Roche Diagnostics GmbH, Mannheim, Germany) with dedicated Cobas 8000 reagents.

Plasma free of hemolysis was adjusted to specified hemoglobin concentrations by adding the supernatant containing the hemolysate. Plasma with increasing hemoglobin concentrations was mixed with an equal volume of Sepharose. Samples were incubated for 30 minutes at room temperature and centrifuged at 1.000 rpm for 5 minutes. Supernatant was removed and hemoglobin concentration determined.

All points were made in triplicate. 100 μl of individual 1:1 Sepharose slurries were added 50 μl plasma with increasing Hb concentration. Incubated for 30 minutes at room temperature under mild agitation, centrifuged at 1000 g for 10 minutes and supernatants isolated. The concentration of hemoglobin and haptoglobin in the supernatant were determined using a Roche Cobas 8000/c701 and Cobas 8000/e802 analyzers (Roche Diagnostics GmbH, Mannheim, Germany) with dedicated Cobas 8000 reagents.

Test of Hemoglobin and Haptoglobin Binding Capacity of Other Non-Mammalian Proteins

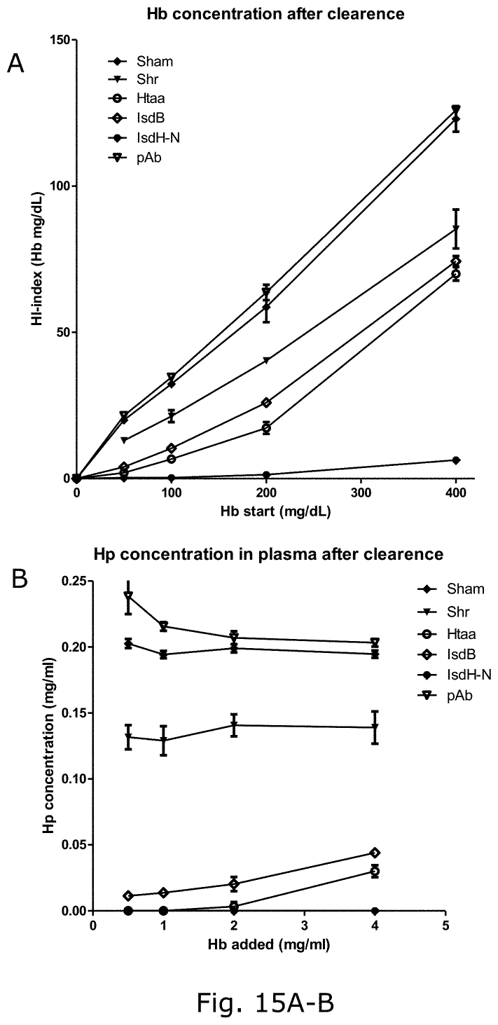

The Hb and Hp concentrations in plasma after the clearance procedure demonstrates that bacterial Hb binding proteins different from IsdH can be used for removal of hemoglobin and haptoglobin (see FIG. 15A-B). Thus, it should be expected that other Hb and HbHp binding non-mammalian proteins can be turned into efficient tools for Hb removal.

Remarkably, the polyclonal antibody against Hb did not clear Hb from plasma. This could indicate that the response in the chickens has primarily been made towards a dominant epitope shielded by Hp binding in plasma. This would suggest that immunization should ideally have been made with the HbHp complex.

Conclusion:

The example shows that a variety of hemoglobin and/or hemoglobin-haptoglobin binding non-mammalian proteins may be used for the removal of hemoglobin from hemolysed samples.

### Example 7: Freezing of the Functionalized Matrix

The IsdH-C-Seph matrix is freeze dried and subsequent to thawing used for the removal of hemoglobin from plasma samples spiked with hemoglobin.

Conclusion:

The example shows that a freeze-dried functionalized matrix can be used for removal of hemoglobin from samples, with sample dilution being reduced to a negligible level.

### Example 8: Conjugation of IsdH to Carboxymethyl Dextran

IsdH is immobilized to carboxymethyl dextran and the affinity of the IsdH-carboxymethyl dextran matrix is compared to that of the IsdH-C-Seph matrix.

Conclusion:

The example shows that the hemoglobin-binding protein may be conjugated to different types of solid supports, while still possessing the capacity to efficiently remove hemoglobin from a sample.

### REFERENCES

WO 2004/036189
Guo et al. (Applied Materials Interfaces (2016), 8, 29734-29741))

1. A method for removal of hemoglobin from a sample, the method comprising:
a) providing a sample comprising hemoglobin,
b) contacting the sample with at least one non-mammalian protein or protein fragment immobilized on a solid support, and
c) separating hemoglobin bound to the at least one non-mammalian protein or protein fragment from the sample,
thereby removing hemoglobin from the sample,
wherein said non-mammalian protein or protein fragment is derived from a unicellular organism and comprises a binding moiety which specifically binds hemoglobin and/or the complex of hemoglobin with haptoglobin.

2-20. (canceled)

21. The method according to claim 1, wherein the binding moiety comprises at least one near iron transporter (NEAT) domain.

22. The method according to claim 21, wherein the at least one NEAT domain comprises a sequence selected from:
i. SEQ ID NO:1, SEQ ID NO:2, SEQ ID NO:3, SEQ ID NO:4, SEQ ID NO:5, SEQ ID NO:6, and combinations thereof, or
ii. a NEAT domain having at least 75% sequence identity to the full-length sequences of any one of SEQ ID NOs:1-6 and combinations thereof.

23. The method according to claim 21, wherein the NEAT domain comprises a sequence selected from:
i. SEQ ID NO:1, SEQ ID NO:2, SEQ ID NO:3, and combinations thereof, or
ii. a NEAT domain having at least 75% sequence identity to the full-length sequence of any one of SEQ ID NOs:1-3 and combinations thereof.

24. The method according to claim 1, wherein the at least one non-mammalian protein or protein fragment comprises a coupling moiety.

25. The method according to claim 24, wherein the coupling moiety is selected from a cysteine residue, an unnatural amino acid, an amino acid linker, an amino group, a consensus sequence or polyethylene glycol (PEG).

26. The method according to claim 24, wherein the coupling moiety is a cysteine residue recombinantly introduced into the at least one non-mammalian protein or protein fragment.

27. The method according to claim 24, wherein the at least one non-mammalian protein or protein fragment is immobilized on the solid support via the coupling moiety.

28. The method according to claim 1, wherein the sample is selected from the group consisting of whole blood, serum, plasma, and red cell lysates.

29. The method according to claim 1, wherein the solid support is of a material selected from the group consisting of plastic, glass, metal, silica, polymers, Sepharose, dextran, carboxymethyl dextran, and combinations thereof.

30. The method according to claim 1, wherein the solid support is in a form selected from the group consisting of a test tube, a resin, a microtiter plate, and a bead.

31. The method according to claim 1, wherein the separation is accomplished by a technique selected from the group consisting of washing, eluting, filtration, centrifugation, magnetic separation, and combinations thereof.

32. The method according to claim 1, wherein the concentration of hemoglobin in the sample after step c) according to claim 1 is reduced to at most 200 mg/dl.

33. A solid support to which is immobilized at least one non-mammalian protein or protein fragment derived from a unicellular organism and comprising a binding moiety which specifically binds hemoglobin and/or the complex of hemoglobin with haptoglobin.

34. The solid support according to claim 33, wherein the binding moiety comprises at least one near iron transporter (NEAT) domain.

35. The solid support according to claim 34, wherein the at least one NEAT domain comprises a sequence selected from:
i. SEQ ID NO:1, SEQ ID NO:2, SEQ ID NO:3, SEQ ID NO:4, SEQ ID NO:5, SEQ ID NO:6, and combinations thereof, or
ii. a NEAT domain having at least 75% sequence identity to the full-length sequences of any one of SEQ ID NOs:1-6 and combinations thereof.

36. The solid support according to claim 33, wherein the solid support is of a material selected from the group consisting of plastic, glass, metal, silica, polymers, Sepharose, dextran, carboxymethyl dextran, and combinations thereof.

37. A container comprising a solid support according to claim 33.

38. A kit for removal of hemoglobin from a sample, the kit comprising:
i. a container according to claim 37, and
ii. instructions for use.

39. A method of using a non-mammalian protein or protein fragment for removal of hemoglobin from a sample comprising, contacting a sample, which contains hemoglobin with a non-mammalian protein or protein fragment derived from a unicellular organism and comprising a binding moiety which specifically binds hemoglobin and/or the complex of hemoglobin with haptoglobin.


SEQUENCE LISTING (24 sequences)

SEQ ID NO: 1 - protein, 128 aa, Staphylococcus aureus
  IsdH N1 (1)..(1): Residues 105-232
DIGPREQVNFQLLDKNNETQYYHFFSIKDPADVYYTKKKAEVELDINTASTWKKFEVYENNQKLPVRLVSYSPVPEDHAYIRFPVSDGTQELKIVSSTQIDDGEETNYDYTKLVFAKPIYNDPSLVKS

SEQ ID NO: 2 - protein, 127 aa, Staphylococcus aureus
  IsdH N2 (1)..(1): Residues 345-471
TADNWRPIDFQMKNDKGERQFYHYASTVEPATVIFTKTGPIIELGLKTASTWKKFEVYEGDKKLPVELVSYDSDKDYAYIRFPVSNGTREVKIVSSIEYGENIHEDYDYTLMVFAQPITNNPDDYVD

SEQ ID NO: 3 - protein, 118 aa, Staphylococcus aureus
  IsdH N3 (1)..(1): Residues 543-660
QLTDLQEAHFVVFESEENSESVMDGFVEHPFYTATLNGQKYVVMKTKDDSYWKDLIVEGKRVTTVSKDPKNNSRTLIFPYIPDKAVYNAIVKVVVANIGYEGQYHVRIINQDINTKDD

SEQ ID NO: 4 - protein, 126 aa, Staphylococcus aureus
  IsdB N1 (1)..(1): Residues 144-269
SAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKT

SEQ ID NO: 5 - protein, 118 aa, Staphylococcus aureus
  IsdB N2 (1)..(1): Residues 341-458
KMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKAN

SEQ ID NO: 6 - protein, 1230 aa, Streptococcus pyogenes
  Shr (1)..(1): Residues 22-1251
QTVKSQEPLVQSQLVTTVALTQDNRLLVEEIGPYASQSAGKEYYKHIEKVIVDNDVYEKSLEGERTFDINYQGIKINANLIKDGKHELTIVNKKDGDILITFIKKGDKVTFISAQKLGTTDHQDSLKKDVLSDKPVPQNQGTQKVVKSGKNTANLSLITKLSQEDGAILFPEIDRYSDNKQIKALTQQITKVTVNGTVYKDLISDSVKDTNGWVSNMTGLHLGTKAFKDGENTIVISSKGFEDITITVTKKDGQIHFVSAKQKQHVTAEDRQSTKLDVTTLEKAIKEADAIIAKESNKDAVKDLAEKLQVIKDSYKEIKDSKLLADTHRLLKDTIESYQAGEVSINNLTEGTYTLNFKANKENSEESSMLQGAFDKRAKLVVKADGTMEISMLNTALGQFLIDFSIESKGTYPAAVRKQVGQKDINGSYIRSEFTMPIDDLDKLHKGAVLVSAMGGQESDLNHYDKYTKLDMTFSKTVTKGWSGYQVETDDKEKGVGTERLEKVLVKLGKDLDGDGKLSKTELEQIRGELRLDHYELTDISLLKHAKNITELHLDGNQITEIPKELFSQMKQLRFLNLRSNHLTYLDKDTFKSNAQLRELYLSSNFIHSLEGGLFQSLHHLEQLDLSKNRIGRLCDNPFEGLSRLTSLGFAENSLEEIPEKALEPLTSLNFIDLSQNNLALLPKTIEKLRALSTIVASRNHITRIDNISFKNLPKLSVLDLSTNEISNLPNGIFKQNNQLTKLDFFNNLLTQVEESVFPDVETLNLDVKFNQIKSVSPKVRALIGQHKLTPQKHIAKLEASLDGEKIKYHQAFSLLDLYYWEQKTNSAIDKELVSIEEYQQLLQEKGSDTVSLLNDMQVDWSIVIQLQKKASNGQYVTVDEKLLSNDPKDDLTGEFSLKDPGTYRIRKALITKKFATQKEHIYLTSNDILVAKGPHSHQKDLVENGLRALNQKQLRDGIYYLNASMLKTDLASESMSNKAINHRVTLVVKKGVPYLEVEFRGIKVGKMLGYLGELSYFVDGYQRDLAGKPVGRTKKAEVVSYFTDVTGLPLADRYGKNYPKVLRMKLIEQAKKDGLVPLQVFVPIMDAISKGSGLQTVFMRLDWASLTTEKAKVVKETNNPQENSHLTSTDQLKGPQNRQQEKTPTSPPSAATGIANLTDLLAKKVTGQSTQETSKTDDTDKAEKLKQLVRDHQTSIEGKTAKDTKTKKSDKKHRSNQQSNGEESSSRYH

SEQ ID NO: 7 - protein, 349 aa, Staphylococcus aureus
  IsdB (1)..(1): Residues 120-468
NTYPILNQELREAIKNPAIKDKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQ

SEQ ID NO: 8 - protein, 574 aa, Staphylococcus aureus
  IsdH (1)..(1): Residues 82-655
NYPAADESLKDAIKDPALENKEHDIGPREQVNFQLLDKNNETQYYHFFSIKDPADVYYTKKKAEVELDINTASTWKKFEVYENNQKLPVRLVSYSPVPEDHAYIRFPVSDGTQELKIVSSTQIDDGEETNYDYTKLVFAKPIYNDPSLVKSDTNDAVVTNDQSSSVASNQTNTNTSNQNISTINNANNQPQATTNMSQPAQPKSSTNADQASSQPAHETNSNGNTNDKTNESSNQSDVNQQYPPADESLQDAIKNPAIIDKEHTADNWRPIDFQMKNDKGERQFYHYASTVEPATVIFTKTGPIIELGLKTASTWKKFEVYEGDKKLPVELVSYDSDKDYAYIRFPVSNGTREVKIVSSIEYGENIHEDYDYTLMVFAQPITNNPDDYVDEETYNLQKLLAPYHKAKTLERQVYELEKLQEKLPEKYKAEYKKKLDQTRVELADQVKSAVTEFENVTPTNDQLTDLQEAHFVVFESEENSESVMDGFVEHPFYTATLNGQKYVVMKTKDDSYWKDLIVEGKRVTTVSKDPKNNSRTLIFPYIPDKAVYNAIVKVVVANIGYEGQYHVRIINQDI

SEQ ID NO: 9 - protein, 400 aa, Trypanosoma brucei
  HpHbR (1)..(1): Residues 1-403
MEKPSCRGAGWAQLLWCYGTCCALLLRLIVEASQAAEGLKTKDEVEKACHLAQQLKEVSITLGVIYRTTERHSVQVEAHKTAIDKHADAVSRAVEALTRVDVALQRLKELGKANDTKAVKIIENITSARENLALFNNETQAVLTARDHVHKHRAAALQGWSDAKEKGDAAAEDVWVLLNAAKKGNGSADVKAAAEKCSRYSSSSTSETELQKAIDAAANVGGLSAHKSKYGDVLNKFKLSNASVGAVRDTSGRGGKHMEKVNNVAKLLKDAEVSLAAAAAEIEEVKNAHETKAQEEMKRNGNPIENESETNSGGNAESQGNGDREDKNDEQQQVDEEETKVENGSSEEGSCCGNESNGPHVMKKRHGVEGPRPVDVVSGFRSYASASFALLSLVRVGMLQ

SEQ ID NO: 10 - protein, 250 aa, Candida albicans
  Rbt51 (1)..(1): Residues 1-250
MMSFSLLSIVSIALAATVSATSDGTNAYTAYPSVAKTASINGFADKIYDQLPECAKECVKQSTSNTPCPYWDTGCLCVMPQFGGAIGDCVAKNCKGKEVDSVESLATSICSSAGVGEPYWMIPSSVSDALAKAANAASATTSVETATKSAAAELATTSDTTIVASTSHESKVAETSVAQQTASTEKSSAAETSRAKETSKAEESSKAEETSVAQSSSSANVASVSAETANAGNMPVIAIGGVIAAFAALI

SEQ ID NO: 11 - protein, 687 aa, Yersinia enterocolitica
  HemR (1)..(1): Residues 1-687
MPRSTSDRFRWSPLSLAIACTLSLAVQAADTSSTQTNSKKRIADTMVVTATGNERSSFEAPMMVTVVEADTPTSETATSATDMLRNIPGLTVTGSGRVNGQDVTLRGYGKQGVLTLVDGIRQGTDTGHLNSTFLDPALVKRVEIVRGPSALLYGSGALGGVISYETVDAADLLLPGQNSGYRVYSAAATGDHSFGLGASAFGRTDDVDGILSFGTRDIGNIRQSDGFNAPNDETISNVLAKGTWRIDQIQSLSANLRYYNNSALEPKNPQTSAASSTNLMTDRSTIQRDAQLKYNIKPLDQEWLNATAQVYYSEVEINARPQGTPEEGRKQTTKGGKLENRTRLFTDSFASHLLTYGTEAYKQEQTPSGATESFPQADIRFGSGWLQDEITLRDLPVSILAGTRYDNYRGSSEGYADVDADKWSSRGAVSVTPTDWLMLFGSYAQAFRAPTMGEMYNDSKHFSMNIMGNTLTNYWVPNPNLKPETNETQEYGFGLRFNDLMMAEDDLQFKASYFDTNAKDYISTGVTMDFGFGPGGLYCKNCSTYSTNIDRAKIWGWDATMTYQTQWFNLGLAYNRTRGKNQNTNEWLDTINPDTVTSTLDVPVANSGFAVGWIGTFADRSSRVSSSGTPQAGYGVNDFYVSYKGQEQFKGMTTTVVLGNAFDKGYYGPQGVPQDGRNAKFFVSYQW

SEQ ID NO: 12 - protein, 1077 aa, Haemophilus influenzae
  HgpA (1)..(1): Residues 1-1077
MTNFRLNVLAYSVMLGLTASVAYAEPTNQPTNQPTNQPTNQPTNQPTNQPTNQPTNQPTNQPTNQPTNQNSNASEQLEQINVSGSTENTDTKAPPKIAETVKTAKKLEKEQAQDVKDLVRYETGITVVEAGRFGNSGFAVRGVEENRVAVQIDGLHQAETISSQGFKELFEGYGNFNNTRNSAEIETLKQVTIRKGADSLKSGSGALGGSVSLDTKDARDYLLNKNYYASYKRGYNTADNQNLNTLTLGGRYKYFDAIAVLTSRKGHELENFGYKNYNDKIQGKTREKADPYRRTQDSALLKIGFQPTENHRFSVVADLYKQTSKGHDFSYTLKPNTQYMTYDEKELRHTNDKVERKNIAFVYENFTETPFWDTLKITYSHQKITTSARTDDYCDGNDKCALAGNPLGMKYNQDNQLVGKDGKSAKYQDINKTQVIKERLPFTKPNGRWRFHKVDWDALKKKYPGVPIYASCLEEDNDPSEFCTYEVKTTKKENTFEINGKRYDLLSEADKNVISDEQRLPTNVSYLFSCDGLNCDKKTILGFKKRRNLLKIFLFEVIEKRCQKYGKTKVKANDQLSGPYLFMPNKKGYQANLWSQRDLTSETKQINLDLTKHLELGKTQHDLSYGGLWSEMEKSMTNLAGDTPLNVKWWAQYPHNCATFLPPSTMTPNAKPTLNPERTSTLCNNVNVFSFLIPVKTKTGALYFINDFRVNNYVAFNLGYRYDRVKYEPEYIPGKTPKIPDDMVTNLYIKTPEFDASKADSDPDELSKKEANAAANIKEIAQPKKFSASSYSFGTTLDPLNWLRLQAKYSKGFRAPTSDEIYFTFKHPDFSIQPNRDLQPETAKTKELSLTVHNDMGYITTSVFDTRYQNFIDLSYQGRRDVHGHSKLIPFHFYQNVNRPNAKVTGFEIASQISLGNITKLFNGFSLSYKYTYQKGRINGNIPMNAIQPRTAVYGVSYVHPDDKYGLDLYISHASAKNAEDTYNMFYKEEGKTDSTIKWRSKSYTTIDLLGYIKPIKNLTLRAGVYNLTNRKYITWDSARSIRPFGTSNMINQDTGLGINRFYAPERNYRMSVQFEF

SEQ ID NO: 13 - protein, 992 aa, Haemophilus influenzae
  HgpB (1)..(1): Residues 1-999
MTNFRLNVLAYSVMLGLTASVAYAEPTNQPTNQPTNQPTNQPTNQPTNQPTNQPTNQNSNASEQLEQINVSGSTENTDTKAPPKIAETVKTAKTLEREQADNIKDIVTYETGVTVVEAGRLAQSGFAIRGVDENRVAINIDGLRQAETLSTQGFKELFEGYGNFNNTRNGAEIETLKEVNITKGADSIKNGSGSLGGSVIYKTKDARDYLINKDYYVSYKKGYATENNQSFNTLTLAGRYKKFDVLVVTTSRNGHELENYGYKNYNDKIQGKKREKADPYKIEQDSTLLKLSFNPTENHRFTFGADLYEHRSRGQDLSYTLKYQKTDPNLAEVDSRHTNDKTKRRNISFSYENFSQTPFWDTLKITYSDQRIKTRARTDEYCDAGVRHCEGTDNPTGLKVTDGKITRRDGSELQFEKKNNTAHSNGETYDFKKFIDTDKKVIEDKLTLKNPNDTWYDCSIFNCENNAKIKVFEGNYSYGYDGKWKEVDLEIKTLNGKKFAKIKDPTNKIKSILPSSPGYLERLWQERDLDTNTQQLNLDLTKDFKTWHIEHNLQYGGSYNTAMKRMVNRAGNDASDVQWWATPTLGYNFYDQPYTCATAYSWNANLCPRVDPEFSYLLPIKTTGKSVYLFDNLVITDYLSFDLGYRYDNIHYQPKYKRGITPKLPDDIVKGLFIPLPKNSNSKPDEVKKNVQQNIDYIAKQNKKYKAHSYSFVSTIDPTSFLRLQLKYSKGFRTPTSDEMYFTFKHPDFTILPNADLKPEIAKTKEIAFTLHNDDWGFISTSLFKTNYKNFIDLIFKGEKDFPLVSGGSSLPFSLYQNINRDNASLKGIEINSKVFLGKMAKFMDGFNLSYKYTYQKGRMNGNIPMNAIQPRTMVYGLGYDHPNHKFGFDFYTTHVASKNPEDTYDIYAKDKKQTDTSIKWRSKSYTILDLIGYVQPIKNLTIRAGVYNLTNRKYITWDSARSIRSFGTSNVIEQSTGLGINRFYAPGRNYK

SEQ ID NO: 14 - protein, 1066 aa, Haemophilus influenzae
  HgpC (1)..(1): Residues 1-1066
MTNFKFTLLARSIAFALNASTAYAAQPTNQPTNQPTNQPTNQPTNQPTNQPTNQDSNLSEQLEQINVSGSTETINVKEKKVGETQISAKKLAKQQASDSRDLVRYETGITVVETGRTGASGYAVRGVDENRVGIMVDGLRQAETLSSQGFKELFEGYGNFNNTRNNIEIENVKTATITKGADSLKSGSGALGGSVIFETKDARDYLIDKDYYVSYKRGYQTMNNQNLKTLTLAGRSKKFDILVVDTTRDGHEIENYDYKIYPNKQADLSAVGPTREKADPYQITRQSTLIKLGFQPNENHRLSVALDDSTLETKGMDLSYAFRPYSQADKEIYGERIINDQSKRKNIQFSYENFSQTPFWDHIKLSYSSQKITNKARSDEYCHQSTCNGVSNPQGLHLVEEKGVYKIVDKDNKDFNYQEDKNNPWSYGKELYNSKNEKISNDVDTEGGALDSVLINCEKLNCEKKKFPIYKEKDEEWKDKYEHEDRDITIKELNGKKYGEISLKKSDSSGFTKYESARFLFPKSFGYSTDFVNDRDLNTNTQQIKLDLDKEFHLWHAQHQLKYGGLYEKTLKSMVNHQYNTAANVQWWADYFFCKKPVNGNRIPAPDHSAYRCKLMNSDIGKDTYLIPVTTKNNVLYFGDNVQLTSWLGLDLNYRYDHVKYLPSYDKNIPVPNGLITGLFKKFKSTDYVYGNKYLVPKGYTNCTYTTDCYKQNFEENLALLLRKTDYKHHSYNLGLNLDPTNWLRVQLKYANGFRAPTSDEIYMTFKHPQFSIQPNTDLKAETSKTKEVAFTFYKNSSYITLNAFQNDYRNFIDLVEVGERPIEEGSVVRYPFHQNQNRDRARVRGIEIASRLEMGDLLEKLQRFPLGYKFTYQKGRIKDNGLHPKYKEFLELNKDEHPEYEAIARKPQPMNALQPTTSVYNIGYDAPSQKWGVDMYITNVAAKKAKDSFNSQWTSMVARKEKIYDTESTVPAKKANGKEVKDSRGLWRNNRYTVIDTIAYWKPIKNLTFTAGVYNLTNKKYLTWDSARSVRHLGTINRVETATGKGLNRLYAPGRNYRMSVQFEF

SEQ ID NO: 15 - protein, 788 aa, Neisseria meningitidis
  HpuB (1)..(1): Residues 23-810
ADPAPQSTQTLNEITVTGTHKTQKLGEEKIRRKTLDKLLVNDEHDLVRYDPGISVVEGGRAGSNGFTIRGVDKDRVAINVDGLAQAESRSSEAFQELFGAYGNFNANRNTSEPENFSEVTITKGADSLKSGSGALGGAVNYQTKSASDYVSEDKPYHLGIKGGSVGKNSQKFSSITAAGRLFGLDALLVYTRRFGKETKNRSTEGDVEIKNDGYVYNPTDTGGPSKYLTYVATGVARSQPDPQEWVNKSTLFKLGYNFNDQNRIGWIFEDSRTDRFTNELSNLWTGTTTSAATGDYRHRQDVSYRRRSGVEYKNELEHGPWDSLKLRYDKQRIDMNTWTWDIPKNYDTNGINGEVYHSFRHIRQNTAQWTADFEKQLDFSKAVWAAQYGLGGGRGDNANSDYSYFAKLYAPKILASNQAKITMLIENRSKYKFAYWNNAFHLGGNDRFRLNAGIRYDKNSSSAKDDPKYTTAIRGQIPHLGSERAHAGFSYGTGFDWRFTKHLHLLAKYSTGFRAPTSDETWLLFPHPDFYLKANPNLKAEKAKNWELGLAGSGKAGNFKLSGFKTKYRDFIELTYMGVSSDDENNPRYAPLSDGTALVSSPVWQNQNRSSAWVKGLEFNGTWNLDSIGLPQGTHAGVNVSYIKGKAKQTNGQETPINALSPWTAVYSLGYDAPSKRWGINAYATRTAAKKPSDTVHSNDDLNNPWPYAKHSKAYTLFDLSAYLNIGKQVTLRAAAYNITNKQYYTWESLRSIREFGTVNRVDNNTHAGIQRFTSPGRSYNFTIEAKF

SEQ ID NO: 16 - protein, 533 aa, Corynebacterium diphtheriae
  HtaA (1)..(1): Residues 37-569
ADSNQCSFNWGIRQSYRHYILKGAAGKTGGQWATQGIGFSGDKTGIDGAFNFTPGKARIDGNSATIPFPGFIHFKGHDHGSGVYLLDMTFSDWKVVTHGSTADILVDYVSYDSDMSNTKDRGPKITGDDVVLATINLNTPADPASGSIDLSGSTTLSPEGAKLFIAYDVGSPLDPTSGTVALDGSCPSPIGPNSDGNGRNGNKKRSVQSISGNFTGFNKEAMAILSETNDTMNAVTIFMDNAGEFLDELDEFNRRGTKPTDNAHASSPESTTASSNISDATRTSPQTQRSAGTTRGSSERIANSSPQCDASSRGVTQAHAAWGLKKSFQSYITGSIAKGQWNLDGVGYSNGEFTFSGASGAVDPQAKSGFVKFGGTMRFSGHHGILDLNISNPEIVFNGATGTLFAQVRSSDMEGKKSDYGRVAIGNLTFSSLNASETAASGKATMTLHPDGAGAFAGFYEAGSDLDPITFDAQLGGAADCSTGTNAAAVPVSGGKESSIPSGKSEGGTSAGYESGAKNFKIRSAATDDSGID

SEQ ID NO: 17 - protein, 173 aa, Corynebacterium diphtheriae
  HtaA CR1 (1)..(1): Residues 42-214
CSFNWGIRQSYRHYILKGAAGKTGGQWATQGIGFSGDKTGIDGAFNFTPGKARIDGNSATIPFPGFIHFKGHDHGSGVYLLDMTFSDWKVVTHGSTADILVDYVSYDSDMSNTKDRGPKITGDDVVLATINLNTPADPASGSIDLSGSTTLSPEGAKLFIAYDVGSPLDPTSG

SEQ ID NO: 18 - protein, 154 aa, Corynebacterium diphtheriae
  HtaA CR2 (1)..(1): Residues 354-507
AHAAWGLKKSFQSYITGSIAKGQWNLDGVGYSNGEFTFSGASGAVDPQAKSGFVKFGGTMRFSGHHGILDLNISNPEIVFNGATGTLFAQVRSSDMEGKKSDYGRVAIGNLTFSSLNASETAASGKATMTLHPDGAGAFAGFYEAGSDLDPITF

SEQ ID NO: 19 - protein, 574 aa, Staphylococcus aureus
  IsdH Y642A mut (1)..(1): Residues 82-655
NYPAADESLKDAIKDPALENKEHDIGPREQVNFQLLDKNNETQYYHFFSIKDPADVYYTKKKAEVELDINTASTWKKFEVYENNQKLPVRLVSYSPVPEDHAYIRFPVSDGTQELKIVSSTQIDDGEETNYDYTKLVFAKPIYNDPSLVKSDTNDAVVTNDQSSSVASNQTNTNTSNQNISTINNANNQPQATTNMSQPAQPKSSTNADQASSQPAHETNSNGNTNDKTNESSNQSDVNQQYPPADESLQDAIKNPAIIDKEHTADNWRPIDFQMKNDKGERQFYHYASTVEPATVIFTKTGPIIELGLKTASTWKKFEVYEGDKKLPVELVSYDSDKDYAYIRFPVSNGTREVKIVSSIEYGENIHEDYDYTLMVFAQPITNNPDDYVDEETYNLQKLLAPYHKAKTLERQVYELEKLQEKLPEKYKAEYKKKLDQTRVELADQVKSAVTEFENVTPTNDQLTDLQEAHFVVFESEENSESVMDGFVEHPFYTATLNGQKYVVMKTKDDSYWKDLIVEGKRVTTVSKDPKNNSRTLIFPYIPDKAVYNAIVKVVVANIGAEGQYHVRIINQDI

SEQ ID NO: 20 - protein, 118 aa, Staphylococcus aureus
  IsdH N3 Y642A mut (1)..(1): Residues 543-660
QLTDLQEAHFVVFESEENSESVMDGFVEHPFYTATLNGQKYVVMKTKDDSYWKDLIVEGKRVTTVSKDPKNNSRTLIFPYIPDKAVYNAIVKVVVANIGAEGQYHVRIINQDINTKDD

SEQ ID NO: 21 - protein, 133 aa, Streptococcus pyogenes
  Shr N1 (1)..(1): Residues 369-501
LTEGTYTLNFKANKENSEESSMLQGAFDKRAKLVVKADGTMEISMLNTALGQFLIDFSIESKGTYPAAVRKQVGQKDINGSYIRSEFTMPIDDLDKLHKGAVLVSAMGGQESDLNHYDKYTKLDMTFSKTVTK

SEQ ID NO: 22 - protein, 160 aa, Streptococcus pyogenes
  Shr N2 (1)..(1): Residues 976-1138
LRDGIYYLNASMLKTDLASESMSNKAINHRVTLVVKKGVPYLEVEFRGIKVGKMLGYLGELSYFVDGYQRDLAGKPVGRTKKAEVVSYFTDVTGLPLADRYGKNYPKVLRMKLIEQAKKDGLVPLQVFVPIMDAISKGSGLQTVFMRLDWASLTTEKAKV

SEQ ID NO: 23 - protein, 604 aa, Staphylococcus aureus
  IsdH N1N2N3-Y642A-Cys (1)..(1): Cys mutant
MGSSHHHHHHSSGLVPRGSHMLENYPAADESLKDAIKDPALENKEHDIGPREQVNFQLLDKNNETQYYHFFSIKDPADVYYTKKKAEVELDINTASTWKKFEVYENNQKLPVRLVSYSPVPEDHAYIRFPVSDGTQELKIVSSTQIDDGEETNYDYTKLVFAKPIYNDPSLVKSDTNDAVVTNDQSSSVASNQTNTNTSNQNTSTINNANNQPQATTNMSQPAQPKSSTNADQASSQPAHETNSNGNTNDKTNESSNQSDVNQQYPPADESLQDAIKNPAIIDKEHTADNWRPIDFQMKNDKGERQFYHYASTVEPATVIFTKTGPIIELGLKTASTWKKFEVYEGDKKLPVELVSYDSDKDYAYIRFPVSNGTREVKIVSSIEYGENIHEDYDYTLMVFAQPITNNPDDYVDEETYNLQKLLAPYHKAKTLERQVYELEKLQEKLPEKYKAEYKKKLDQTRVELADQVKSAVTEFENVTPTNDQLTDLQEAHFVVFESEENSESVMDGFVEHPFYTATLNGQKYVVMKTKDDSYWKDLIVEGKRVTTVSKDPKNNSRTLIFPYIPDKAVYNAIVKVVVANIGAEGQYHVRIINQDIGSGSGSC

SEQ ID NO: 24 - protein, 582 aa, Staphylococcus aureus
ENYPAADESLKDAIKDPALENKEHDIGPREQVNFQLLDKNNETQYYHFFSIKDPADVYYTKKKAEVELDINTASTWKKFEVYENNQKLPVRLVSYSPVPEDHAYIRFPVSDGTQELKIVSSTQIDDGEETNYDYTKLVFAKPIYNDPSLVKSDTNDAVVTNDQSSSVASNQTNTNTSNQNTSTINNANNQPQATTNMSQPAQPKSSTNADQASSQPAHETNSNGNTNDKTNESSNQSDVNQQYPPADESLQDAIKNPAIIDKEHTADNWRPIDFQMKNDKGERQFYHYASTVEPATVIFTKTGPIIELGLKTASTWKKFEVYEGDKKLPVELVSYDSDKDYAYIRFPVSNGTREVKIVSSIEYGENIHEDYDYTLMVFAQPITNNPDDYVDEETYNLQKLLAPYHKAKTLERQVYELEKLQEKLPEKYKAEYKKKLDQTRVELADQVKSAVTEFENVTPTNDQLTDLQEAHFVVFESEENSESVMDGFVEHPFYTATLNGQKYVVMKTKDDSYWKDLIVEGKRVTTVSKDPKNNSRTLIFPYIPDKAVYNAIVKVVVANIGAEGQYHVRIINQDIGSGSGSC